(19) United States
(12) Patent Application Publication
(10) Pub. No.: US 2006/0083784 A1
(43) Pub. Date: Apr. 20, 2006

(54) Amorphous pharmaceutical compositions

(75) Inventors: Francis Ignatious, King of Prussia, PA (US); Linghong Sun, Collegeville, PA (US); Andrew Craig, Tonbridge (GB); David Crowe, Tonbridge (GB); Tim Ho, Tonbridge (GB); Michael Millan, Tonbridge (GB)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/064,890

(22) Filed: Feb. 24, 2005

Related U.S. Application Data
(63) Continuation-in-part of application No. 10/523,835, filed on Feb. 7, 2005.
(60) Provisional application No. 60/401,726, filed on Aug. 7, 2002.

(51) Int. Cl. A61K 9/20; A61K 31/343

(52) U.S. Cl. 424/464; 514/469; 549/467

(57) ABSTRACT

The present invention is directed to use of electrospinning, i.e. the process of making polymer nanofibers from either a solution or melt under electrical forces, to prepare stable, solid dispersions of amorphous drugs in polymer nanofibers. The present invention is also directed to the process of making solid dispersions of amorphous forms and compositions of rosiglitazone and its pharmaceutically acceptable salts.

This application is a continuation in part application of U.S. Ser. No. 10/523,835, filed 7 Feb. 2005, which is the §371 national stage entry of PCT/US 2003/024641, filed 7 Aug. 2003, which claims the benefit of priority from U.S. Ser. No. 60/401,726, filed 7 Aug. 2002.

### FIELD OF THE INVENTION

This invention relates to stabilization of solid dispersions of amorphous drugs in polymeric nanofibers, method of preparation thereof and pharmaceutical compositions containing these nanofibers.

### BACKGROUND

With the advent of combinatorial chemistry and high throughput screening, a great majority of the drug candidates selected for development are highly hydrophobic, exhibiting poor or negligible water solubility. In order to enhance the oral absorption of such poorly water soluble drugs, several formulation strategies such as salt formation, complexation, particle size reduction, prodrug, micellization, and solid dispersions are being extensively studied in the pharmaceutical industry.

Although solid dispersions have been known for the past four decades, there seems to be renewed interest in this technology, as described by Serajudin et al., Journal of Pharmaceutical Sciences, 1999, 88 (10), 1058 and by Habib et al., Pharmaceutical Solid Dispersion Technology, (Technomic, Lancaster, Pa., 2001). Solid dispersions may be defined as the dispersion of one or more active ingredient in an inert carrier or matrix in the solid state prepared by the melting method, the solvent method or the melting-solvent method. Solid dispersions are classified into six major categories: (1) simple eutectic mixtures (2) solid solutions, (3) glass solutions of suspensions, (4) amorphous precipitation of a drug in a crystalline carrier, (5) amorphous precipitation of a drug in a amorphous carrier, and (6) any combination of these groups.

Two currently used methods of forming solid dispersions are fusion and solvent methods. In the fusion method, the drug and the carrier are melted, to above either the melting (softening) point of the higher melting (softening) component, or in some cases to above the melting point of the lower melting component provided the other non-melted component has good solubility in the former. The fused mixture is rapidly quenched and pulverized to produce free flowing powders for capsule filling or tableting. The fusion process requires both the drug and excipient to be thermally stable at the processing temperature.

In the solvent method, the drug and carrier are dissolved in one or more miscible organic solvents to form a solution. Removal of the organic solvent(s) is accomplished by any one or a combination of methods such as solvent evaporation, precipitation by a non-solvent, freeze drying, spray drying, and spray congealing. Among the several draw backs of the solvent method are: use of large volumes of organic solvents, presence of residual organic solvents in the resultant formulation, collection, recycling and/or disposal of organic solvents.

Solid dispersions of poorly soluble drugs prepared by both the fusion and solvent methods usually exhibit higher dissolution rates than the comparative crystalline drug. However, the dissolution rate of the drug may be hindered by dissolution of the carrier, usually a high molecular weight polymer. Therefore solid dispersions are usually prepared from low or moderate molecular weight polymers.

The need still remains to develop a process by which solid dispersions can be made of drugs having an amorphous morphology, that remain stable, and can use higher molecular polymers to aid in the dissolution rates of these drugs.

### BRIEF DESCRIPTION OF THE DRAWINGS

FIG. 1 demonstrates electrospinning of viscous drug/polymer compositions either in solution or in melt form to produce nanofibers.

FIG. 2 shows the X-Ray powder diffraction (XRPD) of electrospun 6-Acetyl-3,4-dihydro-2,2-dimethyl-trans(+)-4-(4-fluorobenzoylamino)-2H-benzo[b]pyran-3-ol hemihydrate fibers during storage up to 161 days at 25° C. Comparison with XRPD of the crystalline compound also shown in the figure, confirms the amorphous nature of the electrospun fiber.

FIG. 3 demonstrates the enhanced in vitro dissolution profiles of electrospun amorphous 6-Acetyl-3,4-dihydro-2,2-dimethyl-trans(+)-4-(4-fluorobenzoylamino)- 2H-benzo[b]pyran-3-ol hemihydrate fibers in comparison to crystalline ones.

FIG. 4 shows the XRPDs of electrospun 3-Hydroxy-2-phenyl-N-[1-phenylpropyl]-4-quinoline carboxamide (Talnetant) fibers during storage up to 120 days at 25° C. For comparison XRPD of the crystalline drug and PVP are included in the figure. The X-ray difractograms show a halo, without any sharp peaks, attesting to the amorphous nature of the electrospun sample.

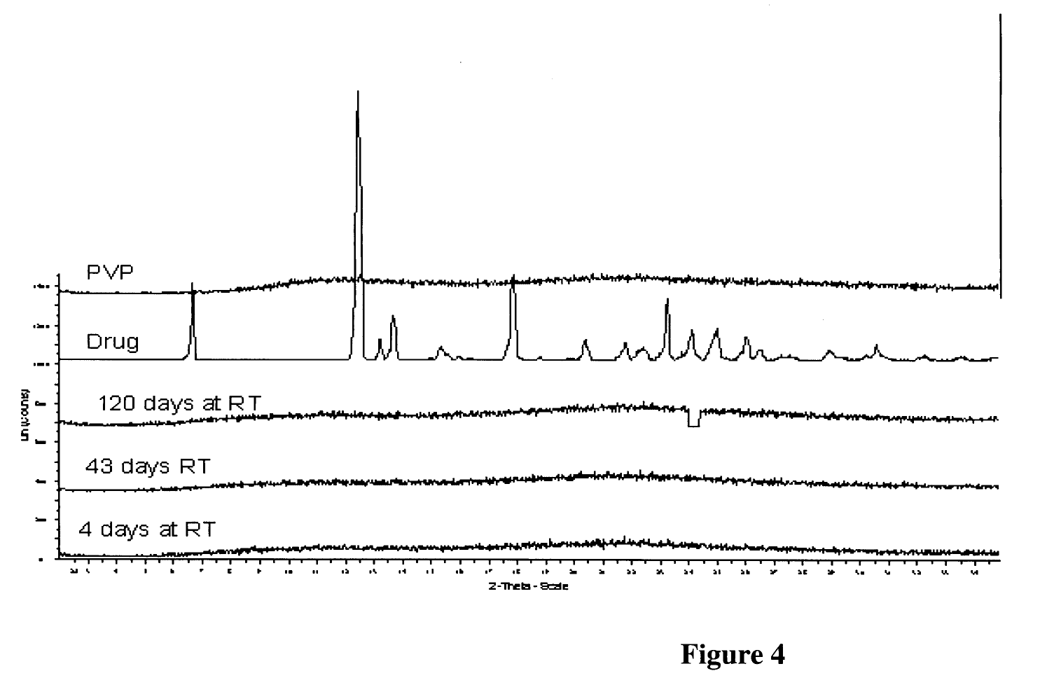

FIG. 5 shows the XRPD of the amorphous solid dispersion of 1:4 wt:wt of rosiglitazone/hydroxypropylmethyl cellulose

FIG. 6 shows the XRPD of the amorphous solid dispersion of 1:2 wt:wt rosiglitazone/PVP

FIGS. 7 and 8 show the XRPD of amorphous rosiglitazone maleate

FIG. 9 shows the XRPD of the amorphous solid dispersion of 1:2 wt:wt rosiglitazone maleate/HPMC

FIG. 10 shows the XRPD of the amorphous solid dispersion of 1:2 wt:wt rosiglitazone maleate/methyl cellulose

FIG. 11 shows the XRPD of amorphous rosiglitazone hydrochloride

FIG. 12 shows the XRPD of the amorphous solid dispersion of 1:2 wt:wt rosiglitazone hydrochloride/PEG

FIG. 13 shows the XRPD of the amorphous solid dispersion of 1:2 wt:wt rosiglitazone hydrochloride/HPMC

FIG. 14 shows the XRPD of the amorphous solid dispersion of 1:1 wt:wt rosiglitazone potassium salt/ethyl cellulose

FIG. 15 shows the XRPD of amorphous rosiglitazone mesylate

FIG. 16 shows the XRPD of the amorphous solid dispersion of 1:2 wt:wt rosiglitazone L(+)-tartrate/HPMC

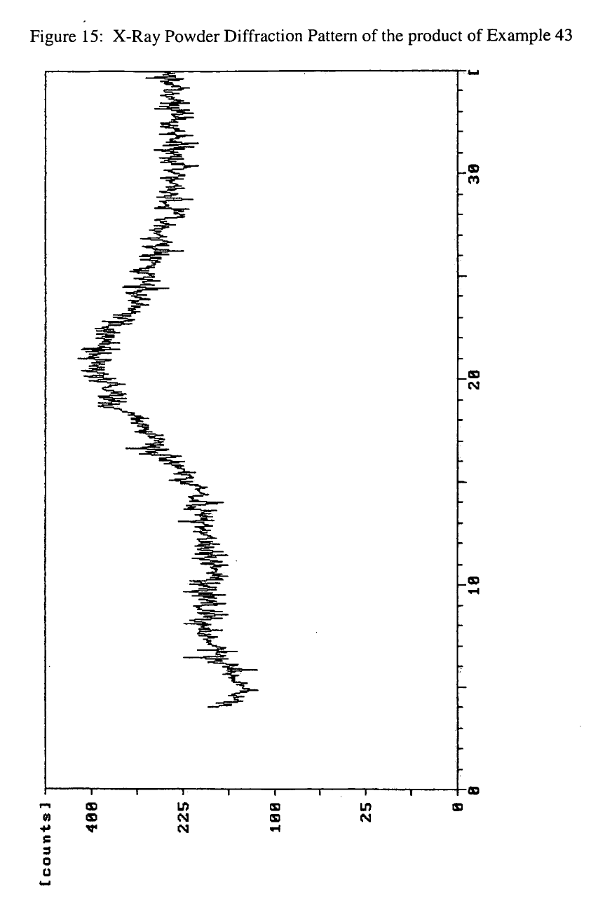

### DETAILED DESCRIPTION OF THE INVENTION

The present invention is directed to the discovery that the technique of electrospinning, i.e. the process of making polymer nanofibers from either a solution or melt under electrical forces, can be used to prepare stable, solid dispersions of an amorphous form of a drug in a polymer nanofibers.

Amorphous solids are disordered materials, which have no long-range order like crystalline materials. Amorphous materials exhibit both compositional and structural disorder. There is a distinguishing difference between compositional disorder and structural disorder. In compositional disorder, atoms are located in an ordered array like in crystalline materials. The spacing of the atoms is equidistant, but only the type of atom is placed randomly. In structural disorder, all bond distances have random lengths and random angles. Therefore there is no long range order, and hence no definite X-ray diffraction patterns. Amorphous solid is a glass in which atoms and molecules exist in a totally non-uniform array. Amorphous solids have no faces and cannot be identified as either habits or polymorphs. Because the properties of amorphous solids are direction independent, these solids are called isotropic. Amorphous solids are characterized by a unique glass transition temperature, the temperature at which it changes from a glass to rubber.

Due to the absence of long-range order, amorphous materials are in an unstable (excited state) equilibrium, resulting in physical as well as chemical instability. The physical instability manifests itself in higher intrinsic aqueous solubility compared to the crystalline drug. The higher solubility of the amorphous drug leads to a higher rate of dissolution, and to better oral bioavailability.

The pharmaceutical industry makes use of the amorphous state of a poorly soluble drug to enhance its aqueous solubility, and its oral bioavailability. However, as stated above, the amorphous state has undesirable physical and chemical instability. This can be overcome by blending the amorphous drug with appropriate polymers, to stabilize the amorphous state, for the desired shelf-life of the drug. It has been reported [Zografi et al, Pharm. Res. 1999, 16, 1722-1728] that the polymer-drug combination should have some specific interaction for stabilization of the amorphous drug.

Amphorous Rosiglitazone

One agent which has demonstrated amorphous characteristics is rosiglitazone. Rosiglitazone is a highly selective agonist for peroxisome proliferator-activated receptor gamma (PPARγ), which is used to treat non-insulin dependent diabetes (NIDDM). Crystalline forms of rosiglitazone (5-[4-[2-(N-methyl-N-(2-pyridyl)amino)ethoxy]-benzyl]-2,4-thiazolidinedione) acid and base salts are well known in the art. The commercial form of rosiglitazone is a crystalline salt, rosiglitazone maleate. Use of rosiglitazone and compositions thereof are generally described in U.S. Pat. No. 5,002,953; U.S. Pat. No. 5,741,803 and US 2002/0177612A1 whose disclosures are incorporated by reference herein.

There is a need for rosiglitazone forms and/or compositions which display improved solubility, particularly over the full pharmacological pH range. Amorphous forms and compositions of rosiglitazone, rosiglitazone maleate and other rosiglitazone salts exhibit such improved solubility characteristics and/or improved dissolution profiles over a wide pharmacological pH range. Additionally, certain compositions of amorphous rosiglitazone or rosiglitazone salts with pharmaceutically acceptable carriers display particularly good solubility and dissolution characteristics, and furthermore have been shown to display good stability characteristics.

The preparation of amorphous rosiglitazone, as a single component, via conventional techniques suffers from a number of disadvantages. Using solution evaporation techniques, one limiting factor is the very poor solubility of rosiglitazone in most suitable processing solvents. This means that few solvent systems are practicable with respect to initial dissolution of the material. Furthermore, for the limited number of solvent systems in which dissolution is possible, the rosiglitazone has a high propensity to crystallise during the evaporation process. Preparation of amorphous rosiglitazone free base by melt techniques, results in high impurity levels due to partial decomposition of the product. Therefore, there remains a need to find additional suitable amorphous forms and amorphous compositions of rosiglitazone.

Compositions of amorphous rosiglitazone free base may be prepared with pharmaceutically acceptable materials such as pharmaceutical excipients. In particular, amorphous rosiglitazone free base in the form of solid dispersions have been prepared with certain pharmaceutically acceptable “carrier” or “matrix” materials, typically, but not exclusively, polymeric materials. The active ingredient is dispersed with the polymer or excipient material so that the rosiglitazone itself does not exist in a discrete crystalline form. Certain amorphous rosiglitazone compositions enhance the solubility of rosiglitazone in both water and organic solvents and stabilise the amorphous rosiglitazone. These compositions are free flowing, easily handleable powders which are potentially suitable for formulation with conventional pharmaceutical excipients, e.g. to prepare tablets.

Rosiglitazone salts have also been prepared successfully in amorphous form as compositions with pharmaceutically acceptable excipients, such as solid dispersions. Solid dispersions of amorphous rosiglitazone salts with pharmaceutically acceptable excipients have the advantage of enhanced stability and solubility. In particular, solid dispersions with certain pharmaceutically acceptable materials have enhanced stability under humid conditions over extended periods of time.

The “material” or “pharmaceutically acceptable material ” or “excipient” or “pharmaceutically acceptable excipient” or “pharmaceutically acceptable carrier” or “polymeric carrier” as used herein, to prepare the non-crystalline compositions of the rosiglitzone may include, but is not limited to: a cellulosic derivative, such as hydroxypropyl methyl cellulose (HPMC), hydroxypropyl cellulose (HPC), methyl cellulose, ethyl cellulose, carboxymethyl cellulose, sodium carboxymethyl cellulose, cellulose acetate phthalate, cellulose acetate butyrate, or hydroxyethyl cellulose; polymethyl methacrylate (PMMA); polymethacrylate; polyvinyl alcohol; polypropylene; polvinylpyrollidone (PVP); polyethylene glycol (PEG); dextrans; dextrins; chitosan; co(lactic/glycolid) copolymers; poly(orthoester); poly(anhydrate); polyvinyl chloride; polyvinyl acetate; ethylene vinyl acetate; lectins; carbopols; silicon elastomers; polyacrylic polymers; maltodextrins; lactose; fructose; inositol; trehalose; maltose; raffinose; or suitable mixtures thereof.

The pharmaceutically acceptable carrier component of the amorphous rosiglitazone compositions may be crystalline or amorphous.

One aspect of this invention is a pharmaceutically acceptable carrier used in combination with rosiglitazone, or a pharmaceutically acceptable salt thereof, which is a cellulosic derivative, such as hydroxypropylmethyl cellulose, methyl cellulose, or ethyl cellulose, or is a suitable mixture thereof.

Examples of compositions of rosiglitazone or salts thereof with a pharmaceutically acceptable carrier include, but are not limited to:

1:2 wt:wt rosiglitazone/hydroxypropylmethyl cellulose solid dispersion.

1:4 wt:wt rosiglitazone/hydroxypropylmethyl cellulose solid dispersion.

1:2 wt:wt rosiglitazone/methyl cellulose solid dispersion.

1:2 wt:wt rosiglitazone/ethyl cellulose solid dispersion.

1:2 wt:wt rosiglitazone/PMMA solid dispersion.

1:2 wt:wt rosiglitazone maleate/hydroxypropylmethyl cellulose solid dispersion.

1:2 wt:wt. rosiglitazone maleate/methyl cellulose solid dispersion.

1:2 wt:wt. rosiglitazone maleate/ethyl cellulose solid dispersion.

1:2 wt:wt. rosiglitazone hydrochloride/hydroxypropylmethyl cellulose solid dispersion

1:2 wt:wt. rosiglitazone potassium salt/hydroxypropylmethyl cellulose solid dispersion

1:1 wt:wt rosiglitazone potassium salt/ethyl cellulose solid dispersion

1:2 wt:wt rosiglitazone mesylate/hydroxypropylmethyl cellulose solid dispersion

1:2 wt:wt rosiglitazone mesylate/ethyl cellulose solid dispersion

1:2 wt:wt rosiglitazone L(+)-tartrate/hydroxypropylmethyl cellulose solid dispersion

The ratio of rosiglitazone or a salt thereof to the pharmaceutically acceptable carrier can be varied over a wide range and depends on the dosage of rosiglitazone required. A suitable range for the ratio of rosiglitazone or a salt thereof to the pharmaceutically acceptable carrier is about 1:33 to about 5:1. However another aspect of the invention is a wt:wt range typically from about 1:5 to about 1:1, suitably from about 1:4.5 to about 1:1.5.

It is also possible to successfully prepare rosiglitazone salts (both acid and base) as single component amorphous forms. The amorphous rosiglitazone salts of the invention are free flowing white powders which may be suitable for incorporation into pharmaceutical formulations.

Suitable pharmaceutical compositions also include mixtures or admixtures of amorphous rosiglitazone salts and pharmaceutically acceptable materials.

The invention provides processes for the preparation of amorphous forms and compositions of rosiglitazone and salts thereof.

The invention also provides the use of amorphous forms and compositions of rosiglitazone and salts thereof in the manufacture of a medicament for the treatment, prophylaxis or both of non-insulin dependent diabetes mellitus.

Additionally, the invention also provides a method for the treatment or prophylaxis of non-insulin dependent diabetes mellitus which comprises administering an effective amount of an amorphous form or composition of rosiglitazone and salts thereof to a human in need of said treatment or prophylaxis.

One method for the preparation of amorphous forms and compositions of rosiglitazone or salts thereof is the solvent evaporation method. This involves, for example, dissolving a mixture of the active ingredient and optionally a pharmaceutically acceptable carrier, in a solvent, or mixture of solvents which may include water, and then removing the solvent by evaporation. The resulting amorphous forms and compositions of rosiglitazone or salts thereof may be used in the preparation of suitable pharmaceutical formulations using conventional methods.

In one embodiment the invention provides a process for preparing amorphous rosiglitazone salt wherein the process comprises:
a) forming a solution of a rosiglitazone salt in one or more organic solvents, or a mixture of organic solvent(s) and water, or water; and
b) removing the solvent by evaporation.

Suitable organic solvents solvents include alcohols, ketones, esters, ethers, nitrites, hydrocarbons, organic acids and chlorinated solvents. Preferably the organic solvents are methanol, ethanol, propan-2-ol, acetone, ethyl acetate, tetrahydrofuran, acetonitrile, toluene, acetic acid or dichloromethane. Even more preferably the organic solvent is selected from methanol, acetone and tetrahydrofuran. For certain less soluble salts, heating may be used if required to achieve solubilization of the rosiglitazone or salt thereof.

In another embodiment the invention provides a process for preparing amorphous rosiglitazone or a salt thereof with particles of a pharmaceutically acceptable carrier where the process comprises:
a) preparing a mixture of a solution of rosiglitazone or a salt thereof and a suspension of the pharmaceutically acceptable carrier, in one or more organic solvents, or a mixture of organic solvent(s) and water, or water; and
b) removing the solvent by evaporation.

In this process the ratio by weight of the rosiglitazone or one of its acid or base salts to acceptable polymeric carrier is typically, in the range of about 1:33 to 5:1, more preferably in the range of about 1:5 to 1:1 and even more preferably in the range of about 1:4.5 to 1:1.5. Suitable organic solvents include alcohols, ketones, esters, ethers, nitrites, hydrocarbons, organic acids and chlorinated solvents. In one embodiment of the invention, the organic solvents are selected from methanol, ethanol, propan-2-ol, acetone, ethyl acetate, tetrahydrofuran, acetonitrile, toluene, acetic acid or dichloromethane. In another embodiment, the organic solvent is selected from methanol, acetone, dichloromethane and tetrahydrofuran. For certain less soluble salts, heating may be used if required to achieve solubilization of the rosiglitazone or salt thereof.

In a further embodiment the invention provides a process for preparing a solid dispersion of amorphous rosiglitazone or a salt thereof and a pharmaceutically acceptable carrier wherein the process comprises:
a) preparing a solution of rosiglitazone or one of its salts and what is typically, but not necessarily, a water soluble polymeric carrier, in one or more organic solvents, or a mixture of organic solvent(s) and water, or water; and
b) removing the solvent by evaporation.

In this process the ratio by weight of the rosiglitazone or one of its acid or base salts to acceptable polymeric carrier is typically, in the range of about 1:33 to 5:1, more preferably in the range of about 1:5 to 1:1 and even more preferably in the range of about 1:4.5 to 1:1.5. Suitable organic solvents include alcohols, ketones, esters, ethers, nitrites, hydrocarbons, organic acids and chlorinated solvents. In one embodiment of the invention the organic solvents are selected from methanol, ethanol, propan-2-ol, acetone, ethyl acetate, tetrahydrofuran, acetonitrile, toluene, acetic acid or dichloromethane. In another embodiment of the invention the organic solvent is selected from methanol, acetone, dichloromethane and tetrahydrofuran. For certain less soluble salts, heating may be used if required to achieve solubilization of the rosiglitazone or salt thereof.

The solvent method is particularly advantageous for the preparation of solid dispersions as the presence of a pharmaceutically acceptable carrier may afford the added benefits of enhancing the solubility of rosiglitazone or a salt thereof in both organic and aqueous solvents, hence facilitating the preparation of a solution (i.e. dissolution ) and also inhibiting crystallisation during the isolation process.

Particularly useful evaporation methods are for example, spray drying, freeze drying and evaporation under reduced pressure.

One evaporation method is spray-drying. This process has been shown to be useful for preparing free-flowing powdery material and is convenient for operation on a commercial scale. Any solvent that will dissolve rosiglitazone or a salt thereof that can be evaporated safely in a spray drying process may be used. Suitable solvents for forming the solution include, but are not limited to, acetone, methanol, propan-2-ol, dichloromethane, tetrahydrofuran and water, or mixtures thereof. Solution concentration will typically be 0.5-50% specifically 2-40% e.g. 3-30%. The concentration that may be employed will only be limited by the dissolution power of the solvent. Spray drying maybe performed, for example, using apparatus supplied by Buchi or Niro. For example, for a Niro S D Micro apparatus the following conditions are appropriate. A Two Fluid nozzle with a diameter (φ) of, e.g. 0.5 mm is suitable, although alternate atomisation methods such as rotary and pressure nozzles can be used. A nozzle position of 0.5 mm above the lowest possible position within the cap is typical. Process gas flow rate is typically 20-30 Kg/h. Solution flow rate may typically be in the range 1-100 ml/min, especially 5-30 ml/min. Inlet temperatures can range from 50-250° C., typically 50-160° C. The inlet temperature and flow rate combination should be suitable to maximise the solvent removal to minimise the risk of solvent trapped in the particle expediting an amorphous to crystalline transition. Spray drying may be combined with further drying, post isolation, in order to assist in this process.

Spray drying processes have been found that can be operated at low temperatures. Inlet temperatures as low as 60-80° C. can be successfully used. This is advantageous for the avoidance of impurity formation.

Another embodiment the present invention provides for a process for preparing the amorphous forms and compositions of rosiglitazone and salts thereof which process comprises:
a) forming a solution of rosiglitazone or a salt thereof in a suitable solvent, optionally mixed with a pharmaceutically acceptable carrier, either as a solution or in suspension; and
b) evaporating the solvent by spray drying, whereby the inlet temperature is in the range of about 60 to about 80° C.

In this process the ratio by weight of the rosiglitazone maleate to acceptable polymeric carrier is typically, in the range of about 1:33 to 5:1, more preferably in the range of about 1:5 to 1:1 and even more preferably in the range of about 1:4.5 to 1:1.5. Suitable solvents include alcohols, ketones, esters, ethers, nitrites, hydrocarbons, organic acids and chlorinated solvents. In one embodiment of the invention the organic solvents are selected from methanol, ethanol, propan-2-ol, acetone, ethyl acetate, tetrahydrofuran, acetonitrile, toluene, acetic acid or dichloromethane. In another embodiment of the invention the organic solvent is selected from methanol, acetone, dichloromethane and water, or mixtures thereof. Heating may be used, if required, to solubilize the rosiglitazone or salts thereof. In one embodiment of the invention a suitable pharmaceutically acceptable carrier includes hydroxypropylmethylcellulose, methylcellulose, ethylcellulose, hydroxypropylcellulose, or polymethylmethacrylate, or a mixture thereof.

Other methods to prepare solid dispersions of amorphous rosiglitazone or a salt thereof and a pharmaceutically acceptable carrier include co-precipitation, where the process comprises:
a) forming a solution of rosiglitazone or a salt thereof and a pharmaceutically acceptable carrier in one or more organic solvents, or a mixture of organic solvent(s) and water, or water; and
b) causing the solid dispersion to precipitate, for example by addition of an anti-solvent, or by changing the pH of the solution.

In this process the ratio by weight of the rosiglitazone maleate to acceptable polymeric carrier is typically, in the range of about 1:33 to 5:1, more preferably in the range of about 1:5 to 1:1 and even more preferably in the range of about 1:4.5 to 1:1.5.. Suitable organic solvents include alcohols, ketones, esters, ethers, nitrites, hydrocarbons, organic acids and chlorinated solvents. In one embodiment the organic solvents are selected from methanol, ethanol, propan-2-ol, acetone, ethyl acetate, tetrahydrofuran, acetonitrile, toluene, acetic acid or dichloromethane. In another embodiment the organic solvent is selected from methanol, acetone, dichloromethane and water, or mixtures thereof. Heating may be used, if required to solubilise the rosiglitazone or salts thereof. In another embodiment, a suitable pharmaceutically acceptable carrier may include the cellulosic derivatives, such as hydroxypropylmethylcellulose, methylcellulose, ethylcellulose, or hydroxypropylcellulose, or polymethylmethacrylate and mixtures thereof.

The amorphous forms and compositions of rosiglitazone and salts thereof can also be prepared by melt processes.

In another embodiment the invention provides a process for preparing a solid dispersion of amorphous rosiglitazone or a salt thereof and a pharmaceutically acceptable carrier by fusion of the drug active into a polymer melt.

In a further aspect the compositions of amorphous rosiglitazone or a salt thereof can be prepared by hot spin melting or hot melt extrusion.

Compositions of the invention in the form of physical mixtures of amorphous rosiglitazone or a salt thereof with one or more pharmaceutically acceptable carriers are prepared by conventional mixing processes, for example by simple mixing, blending or grinding.

Electrospun Fibers

With respect to solid dispersions of pharmaceutically acceptable moieties in general, the electrospun fibers of the present invention are expected to have diameters in the nanometer range, and hence provide a very large surface area. This extremely high surface area can dramatically increase the dissolution rate of the high molecular weight polymeric carrier as well as drug present in them.

A suitable dosage form, such as oral or parenteral forms, including pulmonary administration, may be designed by judicious consideration of polymeric carriers, in terms of their physio-chemical properties as well as their regulatory status. Other pharmaceutically acceptable excipients may be included to ameliorate the stabilization or de-agglomeration of the amorphous drug nanoparticles. The pharmaceutical excipients might also have other attributes, such as absorption enhancers.

Electrospun pharmaceutical dosage forms may be designed to provide any number of dissolution rate profiles, such as rapid dissolution, immediate, or delayed dissolution, or a modified dissolution profile, such as a sustained and/or pulsatile release characteristic.

Taste masking of the active agent may also be achieved by using polymers having functional groups capable of promoting specific interactions with the drug moiety. The electrospun dosage forms may be presented in conventional dosage formats, such as compressed tablets, capsules, sachets or films. These conventional dosage forms may be in the form of immediate, delayed and modified release systems, which can be designed by the appropriate choice of the polymeric carrier with the active agent/drug combination, using techniques well known and described in the art.

It is one embodiment of the present invention to provide drug particles in their amorphous form, embedded homogeneously in the electrospun polymeric nanofibers, such that the drug is readily bioavailable independent of the route of administration.

It is another embodiment of the present invention to provide nanoparticle size drug particles having an amorphous morphology, which are embedded homogeneously within the electrospun polymeric nanofibers.

The starting compound as used herein, may be morphologically either in a crystalline state, or in an amorphous state. As can be seen herein, the present invention provides a novel vehicle which provides a means to allow a crystalline form of a drug to be stabilized in its amorphous form, or to take an amorphous form of a drug and retain its morphology in a controlled environment, i.e. the spun fibers. This can be used as noted, as a means to increase the surface area (nanoparticle size, etc.) and to improve its dissolution rate characteristics.

Electrospinning, also referred to as electrostatic spinning, is a process of producing fibers, with diameters in the range of 100 nm. The process consists of applying a high voltage to a polymer solution or melt to produce a polymerjet. As the jet travels in air, the jet is elongated under repulsive electrostatic force to produce nanofibers. The process has been described in the literature since the 1930. A variety of polymers both natural and synthetic having optimal characteristics have been electrospun under appropriate conditions to produce nanofibers, (see Reneker et al., Nanotechnology, 1996, 7, 216). Different applications have been suggested for these electrospun nanofibers, such as air filters, molecular composites, vascular grafts, and wound dressings.

U.S. Pat. No. 4,043,331, is intended for use as a wound dressing whereas U.S. Pat. No. 4,044,404, and U.S. Pat. No. 4,878,908 are tailored towards creating a blood compatible lining for a prosthetic device. All of the disclosed water insoluble polymers are not pharmaceutically acceptable for use herein, however the water soluble polymers disclosed are believed to be pharmaceutically acceptable. None of the preparations in these patents disclose a working example of an electrospun fiber with an active agent. The patents claim the use of enzymes, drugs and/or active carbon on the surface of the nanofibers, prepared by immobilizing the active moieties so that they act at the site of application and “do not percolate throughout the body”.

EP 542514, U.S. Pat. No. 5,311,884 and U.S. Pat. No. 5,522,879 pertain to use of spun fibers for a piezoelectric biomedical device. The piezoelectric properties of fluorinated polymers, such as those derived from a copolymer of vinylidene fluoride and tetrafluoroethylene are not considered pharmaceutically acceptable polymers for use herein.

U.S. Pat. No. 5,024,671 uses the electrospun porous fibers as a vascular graft material, which is filled with a drug in order to achieve a direct delivery of the drug to the suture site. The porous graft material is impregnated (not electrospun) with the drug and a biodegradable polymer is added to modulate the drug release. The vascular grafts are also made from non-pharmaceutically acceptable polymers, such as the polyterafluorethylene or blends thereof.

U.S. Pat. No. 5,376,116, U.S. Pat. No. 5,575,818, U.S. Pat. No. 5,632,772, U.S. Pat. No. 5,639,278 and U.S. Pat. No. 5,724,004 describe one form or another of a prosthetic device having a coating or lining of an electrospun non-pharmaceutically acceptable polymer. The electrospun outer layer is post-treated with a drug such as disclosed in the '116 patent (for breast prosthesis). The other patents describe the same technology and polymers but apply the technique to other applications, such as endoluminal grafts or endovascular stents.

Consequently, the present invention is the first to produce an electrospun composition of a pharmaceutically acceptable polymer in which one or more pharmaceutically acceptable active agents or drugs are stabilized in their amorphous form. The homogenous nature of this process produces a quantity of fibers which allow for nanoparticles of drugs to be dispersed throughout. The size of particle, and quality of dispersion provide for a high surface area of drug. One use of the increased surface area of drug is improved bioavailability in the case of a poorly water soluble drug. Other uses would be for decreased drug-drug or enzymatic interactions.

Yet another use of the present invention is to delay the release of drugs in the gastrointestinal tract by using pH sensitive polymers, such as the Eudragit group of polymers by Rohm, in particular the Eudragit L100-55 polymer.

The present invention is therefore directed to use in any form of an electrospun drug/polymer combination, wherein the drug is stabilized in the amorphous form; and another wherein the resulting drug/polymer combination provides for enhanced bioavailability of the poorly soluble drug or to modify the absorption profile of the drug(s). The modification of the rate of release of the active compound when incorporated within the polymeric fibers may be increased or decreased. The resulting bloavailability of the active agent may also be increased or decreased relative to the immediate release dosage form.

While the application of the electrospun process may be of use for incorporation of a pharmaceutically acceptable drug for topical delivery, a preferred route of administration is likely to be oral, intravenous, intramuscular, or inhalation.

A pharmaceutically acceptable agent, active agent or drug as defined herein follows the guidelines from the European Union Guide to Good Manufacturing Practice: Any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure and function of the body. Preferably, their use is in a mammal, more preferably a human. The pharmacological activity may be prophylactic or for treatment of a disease state. The pharmaceutical compositions described herein may optionally comprise one or more pharmaceutically acceptable active agents or ingredients distributed within.

As used herein the terms “agent”, “active agent”, “drug moiety” or “drug” are used interchangeably.

The term “prophylaxis” is intended to mean prevention, or inhibition, or delay of onset of a disease condition or disease state in a mammal. It need not be a 100% prevention or inhibition of said condition or disease, e.g. it may be a delay in the disease condition occurring in said mammal, particularly when the mammal is found to be predisposed to having such disease condition, but has not yet been diagnosed as having it.

The term “treatment” is intended to mean the complete or partial amelioration, or reduction of the symptoms, or reduction of the severity of the symptoms, or reduction of the incidence of the symptoms, or any other change in the condition of the patient, which improves the therapeutic outcome in the mammal that is affected, at least in part, by the disease and includes, but is not limited to, modulating the disease condition; and/or alleviating the disease condition.

An “effective amount” or “therapeutically effective amount” or “prophylactically effective amount” is intended to mean that amount of compound or composition of the invention that, when administered to a mammal in need of such treatment or prophylaxis, is sufficient to effect the desired treatment or prophylaxis of the disease state, or is sufficient to result in the prevention of, or delay of onset of the disease, or recovery from the disease. Further, a prophylactically or therapeutically effective amount with respect to a compound or composition of the invention includes that amount alone, or in combination with other agents to effect such treatment or prophylaxis thereof.

Water solubility of the active agent is defined by the United States Pharmacoepia. Therefore, active agents which meet the criteria of very soluble, freely soluble, soluble and sparingly soluble as defined therein are encompassed this invention. It is believed that the electrospun polymeric composition, which most benefits those drugs, are those which are insoluble or sparingly soluble. However, as the electrospun polymeric composition produces, or stabilizes an amorphous form of the drug, the solubility of the drug may not be as important than if it were in a crystalline state.

The fibers of this invention will contain high molecular weight polymeric carriers. These polymers, by virtue of their high molecular weight, form viscous solutions that can produce nanofibers, when subjected to an electrostatic potential. The nano fibers spun electostatically may have a very small diameter. The diameter may be as small as 0.1 nanometers, more typically less than 1 micron. This provides a high surface area to mass ratio. The fiber may be of any length, and it may include particles which vary from the more traditional spun cylindrical shape such as drop-shaped or flat.

Suitable polymeric carriers can be preferably selected from known pharmaceutical excipients. The physico-chemical characteristics of these polymers dictate the design of the dosage form, such as rapid dissolve, immediate release, delayed release, modified release such as sustained release, or pulsatile release etc.

The delivery rate of the active agent can be controlled by varying the choice of the polymer used in the fibers, the concentration of the polymer used in the fiber, the diameter of the polymeric fiber, and/or the amount of the active agent loaded in the fiber.

Suitable drug substances can be selected from a variety of known classes of drugs including, for example, analgesics, anti-inflammatory agents, anthelmintics, anti-arrhythmic agents, antibiotics (including penicillins), anticoagulants, antidepressants, antidiabetic agents, antiepileptics or anticonvulsants (also referred to as neuroprotectants, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, NK3 receptor antagonists, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, PDE IV inhibitors, vasodilators and xanthines.

Preferred drug substances include those intended for oral administration and intravenous administration. A description of these classes of drugs and a listing of species within each class can be found, for example, in Martindale, The Extra Pharmacopoeia, Twenty-ninth Edition, The Pharmaceutical Press, London, 1989, the disclosure of which is hereby incorporated herein by reference in its entirety. The drug substances are commercially available and/or can be prepared by techniques known and described in the art.

As noted, the electrospun composition may also be able to taste mask the many bitter or unpleasant tasting drugs, regardless of their solubility. Suitable active ingredients for incorporation into fibers of the present invention include the many bitter or unpleasant tasting drugs including but not limited to the histamine H₂-antagonists, such as, cimetidine, ranitidine, famotidine, nizatidine, etinidine; lupitidine, nifenidine, niperotidine, roxatidine, sulfotidine, tuvatidine and zaltidine; antibiotics, such as penicillin, ampicillin, amoxycillin, and erythromycin; acetaminophen; aspirin; caffeine, dextromethorphan, diphenhydramine, bromopheniramine, chloropheniramine, theophylline, spironolactone, NSAIDS's such as ibuprofen, ketoprofen, naprosyn, and nabumetone; 5HT₄ inhibitors, such as granisetron, or ondansetron; seratonin re-uptake inhibitors, such as paroxetine, fluoxetine, and sertraline; vitamins such as ascorbic acid, vitamin A, and vitamin D; dietary minerals and nutrients, such as calcium carbonate, calcium lactate, etc., or combinations thereof.

Suitably, the above noted active agents, in particular the anti-inflammatory agents, may also be combined with other active therapeutic agents, such as various steroids, decongestants, antihistamines, etc., as may be appropriate in either the electrospun fiber or in the resulting dosage form.

Suitable active agents for use in the electrospun polymeric fibers herein are 6-Acetyl-3,4-dihydro-2,2-dimethyl-trans(+)-4-(4-fluorobenzoylamino)-2H-benzo[b]pyran-3-ol hemihydrate, 3-Hydroxy-2-phenyl-N-[1-phenylpropyl]-4-quinoline carboxamide (Talnetant), rosiglitazone, carvedilol, hydrochlorothiazide, eprosartan, indomethacin, nifedipine, naproxen, ASA, and ketoprofen, or those described in the Examples section herein.

The relative amount of fiber forming material (primarily the polymeric carrier) and the active agent that may be present in the resultant fiber may vary. In one embodiment the active agent comprises from about 1 to about 50% w/w of the fiber when electrospun, preferably from about 35 to about 45% w/w.

DNA fibers have also been used to form fibers by electrospinning, Fang et al., J. Macromol. Sci.-Phys., B36(2), 169-173 (1997). Incorporation of a pharmaceutically acceptable active agent, such as a biological agent, a vaccine, or a peptide, with DNA, RNA or derivatives thereof as a spun fiber is also within the scope of this invention.

The fiber forming characteristics of the polymer are exploited in the fabrication of nanofibers. Hence, molecular weight of the polymer is one of the single most important parameter for choice of polymer.

Another important criteria for polymer selection is the miscibility between the polymer and the drug. It may be theoretically possible to ascertain the miscibility's by comparing the solubility parameters of the drug and polymer, as described by Hancock et al, in International Journal of Pharmaceutics, 1997, 148, 1.

Another important criteria for polymer selection is its ability to stabilize the amorphous drug. It has been reported by Hancock et al, in Journal of Pharmaceutical Sciences, 1997, 86,1; that stable drug/polymer compositions should have glass transition temperatures (Tg) above the storage temperature. If the Tg of the drug/polymer combination is lower than the storage temperature, the drug will exist in the rubbery state, and will consequently be prone to molecular mobility and crystallisation. An example of this is the polymer poly(ethylene oxide) which is a semicrystalline/crystalline polymer. It has been shown that at least some crystalline drugs spun in such a polymer, having an amorphous morphology initially, will over time crystallize out.

Representative examples of amorphous polymers for use herein in electrospinning applications include, but are not limited to, polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and methacrylic acid copolymers and their derivatives such as are found in the Eudragit family of polymers available from Rohm Pharma, poly(alpha-hydroxy acids) and its copolymers such poly(alpha-aminoacids) and its copolymers, poly(orthoesters), polyphosphazenes, polyethyloxazolines, poly(phosphoesters), and or combinations thereof.

The polymers, poly(ε-caprolactone), poly(lactide-co-glycolide), polyanhydrides, poly(ethylene oxide), are crystalline or semicrystalline polymers.

Most of these pharmaceutically acceptable polymers are described in detail in the Handbook of Pharmaceutical excipients, published jointly by the American Pharmaceutical association and the Pharmaceutical society of Britain.

Preferably, the polymeric carriers are divided into two categories, water soluble polymers useful for immediate release of the active agents, and water insoluble polymers useful for controlled release of the active agents. It is recognized that combinations of both carriers may be used herein. It is also recognized that several of the polyacrylates are pH dependent for the solubility and may fall into both categories.

Water soluble polymers include but are not limited to, polyvinyl alcohol, polyvinyl pyrrolidone, hyaluronic acid, alginates, carragenen, cellulose derivatives such as carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch and its derivatives such as hydroxyethyl starch, sodium starch glycolate, dextrin, chitosan and its derivatives, albumen, zein, gelatin, and collagen.

A suitable water soluble polymer for use herein is polyvinylpyrrolidone, or polyvinylpyrrolidone and its copolymer with polyvinylacetate.

Water insoluble polymers include but are not limited to, polyvinyl acetate, methyl cellulose, ethylcellulose, noncrystalline cellulose, polyacrylates and its derivatives such as the Eudragit family of polymers available from Rohm Pharma (Germany), poly(alpha-hydroxy acids) and its copolymers such as poly(alpha-aminoacids) and its copolymers, poly(orthoesters), polyphosphazenes, and poly(phosphoesters).

The acrylic polymers of the Eudragit family are well known in the art and include a number of different polymers, ranging from Eudragit L100-55 (the spray dried form of Eudragit L30D), Eudragit L30D, Eudragit L100, Eudragit S 100, Eudragit 4135F, Eudragit E100, Eudragit EPO (powder form of E100), Eudragit RL30D, Eudragit RL PO, Eudragit RL 100, Eudragit RS 30D, Eudragit RS PO, Eudragit RS 100, Eudragit NE 30 D, and Eudragit NE 40 D.

These pharmaceutically acceptable polymers and their derivatives are commercially available and/or be prepared by techniques known in the art. By derivatives it is meant, polymers of varying molecular weight, modification of functional groups of the polymers, or co-polymers of these agents, or mixtures thereof.

Further, two or more polymers can be used in combination to form the fibers as noted herein. Such combination may enhance fiber formation or achieve a desired drug release profile. One suitable combinations of polymers includes polyethyleneoxide and polycaprolactone.

Preferably, the polymer of choice is an amorphous polymer, such as but not limited to: polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives such as the Eudragit family of polymers available from Rohm Pharma, such as Eudragit L100-55, poly(alpha-hydroxy acids), poly(alpha-aminoacids) and its copolymers, poly(orthoesters), polyphosphazenes, and poly(phosphoesters). The preferred polymers are ones with functional groups capable of promoting specific interaction with the active agent to help stabilize the amorphous form of the agent. Suitable polymers are PVP and PVP with copolymers or the Eudgragit group of polymers as described herein.

The choice of polymers taken with the active agent may provide suitable taste masking functions for the active agents. For instance, use of an ionic polymer of contrasting charge, such as a cationic polymer complexed with an anionic active agent, or an anionic polymer complexed with a cationic active agent may produce the desired results.

Addition of a second taste masking agent, such as a suitable cyclodextrin, or its derivatives may also be used herein.

The polymeric composition may be electrospun from a solvent base or neat (as a melt). Solvent choice is preferably based upon the solubility of the active agent. Suitably, water is the best solvent for a water soluble active agent, and polymer. Alternatively, water and a water miscible organic solvent may used. However, it is necessary to use an organic solvent to prepare a homogenous solution of the drug with polymer when the drug is non-water soluble, or sparingly soluble.

It is recognized that these polymeric compositions which are spun neat may also contain additional additives such as, plasticizers, and antioxidants. The plasticizers are employed to assist in the melting characteristics of the composition. Exemplary of plasticizers that may be employed in the coatings of this invention are triethyl citrate, triacetin, tributyl citrate, acetyl triethyl citrate, acetyl tributyl citrate, dibutyl phthalate, dibutyl sebacate, vinyl pyrrolidone and propylene glycol.

Suitably, the solvent of choice is a GRASS approved organic solvent, although the solvent may not necessarily be “pharmaceutically acceptable” one, as the resulting amounts may fall below detectable, or set limits for human consumption they may be used. It is suggested that ICH guidelines be used for selection.

Suitable solvents for use in the electrospinning process include, but are not limited to acetic acid, acetone, acetonitrile, methanol, ethanol, propanol, ethyl acetate, propyl acetate, butyl acetate, butanol, N,N dimethyl acetamide, N,N dimethyl formamide, 1-methyl-2-pyrrolidone, dimethyl sulfoxide, diethyl ether, diisopropyl ether, tetrahydrofuran, pentane, hexane, 2-methoxyethanol, formamide, formic acid, hexane, heptane, ethylene glycol, dioxane, 2-ethoxyethanol, trifluoroacetic acid, methyl isopropyl ketone, methyl ethyl ketone, dimethoxy propane, methylene chloride etc., or mixtures thereof.

A preferred solvent is ethanol, acetone, n-vinylpyrrolidone, dichloromethane, acetonitrile, tetrahydrofuran or a mixture of these solvents.

The solvent to polymeric composition ratio is suitably determined by the desired viscosity of the resulting formulation.

For electrospinning of a pharmaceutical polymeric composition, key parameters are viscosity, surface tension, and electrical conductivity of the solvent/polymeric composition.

By the term “nanoparticulate drug” as used herein, is meant, nanoparticule size of an active agent within the electrospun fiber, as opposed to a nanoparticule size of the resulting fibers themselves.

The polymeric carriers may also act as surface modifiers for the nanoparticulate drug. Therefore, a second oligomeric surface modifier may also be added to the electrospinning solution. All of these surface modifiers may physically adsorb to the surface of the drug nanoparticles, so as to prevent them agglomerating.

Representative examples of these second oligomeric surface modifier or excipients, include but are not limited to: Pluronics® (block copolymers of ethylene oxide and propylene oxide), lecithin, Aerosol OT™ (sodium dioctyl sulfosuccinate), sodium lauryl sulfate, Tween™, such as Tween 20, 60 & 80, Span™, Arlacel™, Triton X-200, polyethylene glycols, glyceryl monostearate, Vitamin E-TPGS™ (d-alpha-tocopheryl polyethylene glycol 1000 succinate), sucrose fatty acid esters, such as sucrose stearate, sucrose oleate, sucrose palmitate, sucrose laurate, and sucrose acetate butyrate etc.

Triton X-200 is polyethylene glycol octylphenyl ether sulfate ester sodium salt; or polyethylene glycol octylphenyl ether sulfate sodium salt. Span and Arlacel are synonyms for a sorbitan fatty acid ester as defined in the Handbook of Pharmaceutical Excipients, and Tween is also a synonym for polyoxyethylene sorbitan fatty acid esters.

Surfactants are added on a weight/weight basis to the drug composition. Suitably, the surfactants are added in amounts of up to 15%, preferably about 10%, preferably about 5% or less. Surfactants can lower the viscosity and surface tension of the formulation, and in higher amounts can adversely effect the quality of the electrospun fibers.

The surfactant selection may be guided by HLB values but is not necessarily a useful criteria. While HLB surfactants have been utilised herein, such as Tween™ 80 (HLB=10), Pluronic F68 (HLB=28), and SDS (HLB>40), lower HLB value surfactants, such as Pluronic F92 may also be used.

Another pharmaceutically acceptable excipients may be added to the electrospinning composition. These excipients may be generally classified as absorption enhancers, flavouring agents, dyes, etc.

The polymeric carriers or the second oligomeric surface modifiers, if appropriately chosen, may themselves act as absorption enhancers, depending on the drug. Suitable absorption enhancers for use herein, include but are not limited to, chitosan, lecithin, lectins, sucrose fatty acid esters such as the ones derived from stearic acid, oleic acid, palmitic acid, lauric acid, and Vitamin E-TPGS, and the polyoxyethylene sorbitan fatty acid esters.

Use of the electrospun composition herein may be by conventional capsule or tablet fill as well known in the art. Alternatively, the fibers may be ground, suitably by cryogenic means, for compression into a tablet or capsule, for use by inhalation, or parenteral administration. The fibers may also be dispersed into an aqueous solution, which may then be directly administered by inhaled or given orally. The fibers may also be cut, optionally milled, and processed as a sheet for further administration with agents to form a polymeric film, which may be quick-dissolving.

An alternative electrospinning process for making the pharmaceutical compositions described herein is also possible. The Examples herein electrostatically charge the solution whereas the pharmaceutical composition may also be ejected from a sprayer onto a receiving surface that is electrostatically charged and placed at an appropriate distance from the sprayer. As jet travels in air from the sprayer towards the charged collector, fibers are formed. The collectors can be either a metal screen, or in the form of a moving belt. The fibers deposited on the moving belt are continuously removed and taken away.

The following examples illustrate the invention.

General Procedure for Electrospinning

A solution of the drug and polymer in a suitable organic solvent is electrospun using the following electrospinning set up. The solution to be electrospun is taken in a 25 ml glass vessel having a 0.02 mm capillary outlet at the bottom and two top inlets, one for applying a positive He pressure and the other for introducing the electrode through a rubber septum. The electrode is connected to the positive terminal of a high voltage power supply (Model ES30P/M692, Gamma High Voltage Research Inc., Fla.). The ground from the high voltage power supply is connected to a stainless steel rotating drum, which acts the collector for the fibers. A voltage of 18-25KV is applied to the polymer solution through the electrode which reaches the bottom of the glass vessel. This high voltage creates a monofilament from the capillary outlet and the monofilament is further splayed to form nanofibers. The inlet He pressure varying from 0.5-2 psi is adjusted to maintain a constant feed of liquid to the capillary tip, in order to produce continuous electrospinning and to prevent the formation of excess liquid droplets, which might simply fall off from the capillary. The rotating drum is kept a distance of 15-25 cm from the positive electrode. The dry fibers collected on the drum is peeled off and harvested.

Materials

Polyvinylpyrrolidone (PVP), molecular weight 1.3 M, available from Sigma-Aldrich Chemicals (St. Louis, Mo.) and polyvinylpyrrolidone-co-polyvinylacetate (Kolloidon VA-64), available from BASF, Eudragit L100 55 (Rohm Pharma), polyethylene oxide as POLYOX WSR 1105 (Union Carbide) are used for experiments. Drug substances such as, rosiglitazone, carvedilol, eprosartan, hydrochlorothiazide, indomethacin, nifedipine, ketoprofen, and naproxen are available commercially from the manufacturer or from various catalogs, such as Sigma-Aldrich.

Methods

Drug Content

Drug content in the electrospun samples were determined by an appropriate HPLC method. A weighed amount of electrospun fibers, is dissolved in a solvent and analyzed by Agilent 1100 HPLC system having a C18 column.

In vitro Dissolution Assay

The equipment used for this procedure is a modified USP 4, the major differences being: 1. low volume cell. 2. stirred cell. 3. retaining filters which are adequate at retaining sub micron material. The total run time is 40 minutes. 2.5 mg of drug (weigh proportionally more formulated material).

Flow Cell Description: Swinnex filter assemblies obtained from Millipore, having 0.2 micron Cellulose Nitrate membranes. (Millipore, Mass.) as internal filters. The internal volume of the cell is approximately 2 ml. A Small PTFE stirrer customized to fit the Swinnex assembly (Radleys Lab Equipment Halfround Spinvane F37136) is used. The dissolution medium at a flow rate of 5 ml/min is used. The whole set up is placed at a thermostat of 37° C. The drug concentration is measured by passing the eluent through a UV detector having a flow cell dimension of 10 mm. The UV detection is carried out at an appropriate wavelength for the drug.

Determination of Extent of Drug Solubility

The experimentation is designed to evaluate drug dissolution rate. As such it is unlikely with poorly soluble drugs, and with water as the dissolution medium, that 100% of the drug will dissolve in the 40 minute duration of the test. To determine the extent of drug solubility over this period one collects all 200 ml of solution that elutes from the dissolution cell. Using a conventional UV spectrophotometer, this solution is compared against a reference solution of 2.5 or 4 mg of active agent dissolved in a suitable medium.

Amorphicity and its Stability Over Time

The amorphous nature of the drug in the formulation and its stability on ageing at 25° C. and zero humidity, was determined by XRPD. The instrument is a Bruker D8 AXS Diffractometer. Approximately 30 mg of sample is gently flattened on a silicon sample holder and scanned at from 2-35 degrees two-theta, at 0.02 degrees two-theta per step and a step time of 2.5 seconds. The sample is rotated at 25 rpm to reduce preferred orientation. Generator power is set at 40 mA and 40 kV.

The amorphous nature of the drug was also confirmed by MDSC (TA instruments, New Castle, Del.). The samples in hermetically sealed aluminium pans were heated from 0 to 200, or to 250° C. at 2° C./min at a modulation frequency of ±0.159° C. every 30 seconds.

### EXAMPLE 1

### Preparation of amorphous 6-Acetyl-3,4-dihydro-2,2-dimethyl-trans(+)-4-(4-fluorobenzoylamino)-2H-benzo[b]pyran-3-ol hemihydrate (Compound I) by electrospinning.

Various samples shown in Table 1, were prepared by dissolving the title compound and PVP in ethanol. This solution was electrospun using the set up described in the experimental section above.

TABLE 1

| Ingredients | Sample 1.1 | Sample 1.2 | Sample 1.3 |
|---|---|---|---|
| Compound I | 300 mg | 400 mg | 2 g |
| PVP | 600 mg | 600 mg | 3 g |
| Ethanol used | 10 ml | 7 ml | 40 ml |
| Surfactant (Tween 80) | | 50 mg | none |
| Yield (g) | 400 mg | n/a | 4 g |
| Drug content determined | 37.3% | 37.1% | 33.3% |
| HPLC | | | |

XRPD of the Electrospun Compound I, Sample 1.2

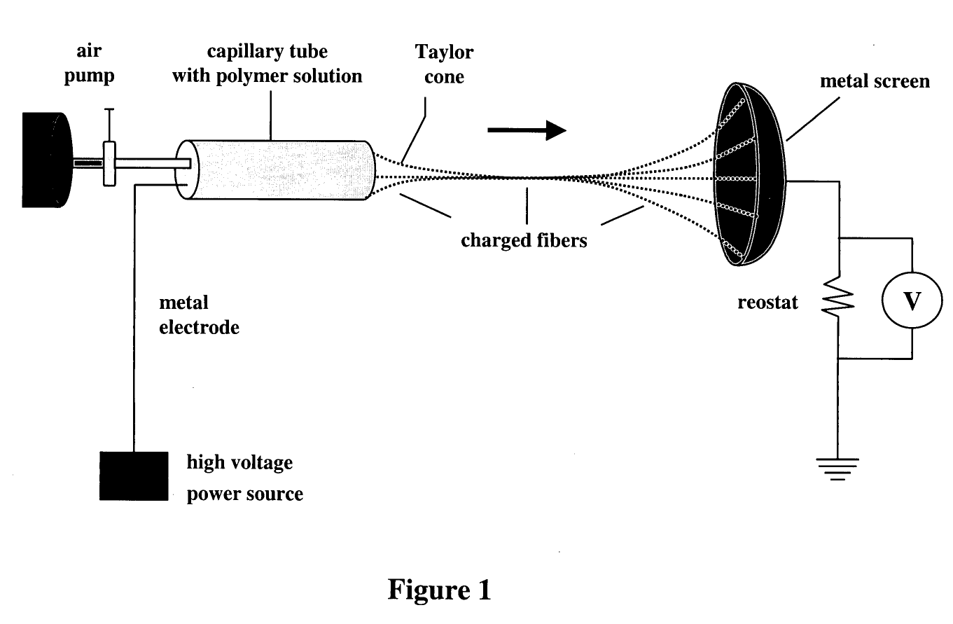

XRPDs of the electrospun sample 1.2 after storage at 25° C. and zero humidity for several days up to 161 days, show the sample to be amorphous. FIG. 1 compares the XRPDs of sample 1.2 stored for 45, 84, 133 and 161 days, along the XRPD of crystalline drug and PVP.

Thermal Analysis of Samples 1.2 and 1.3

Crystalline Compound I exhibits crystalline melting endotherm at 145° C., whereas the sample 1.2 and sample 1.3 do not have a crystalline melting endotherm, when heated from 0 to 200° C.

In vitro Dissolution Rates

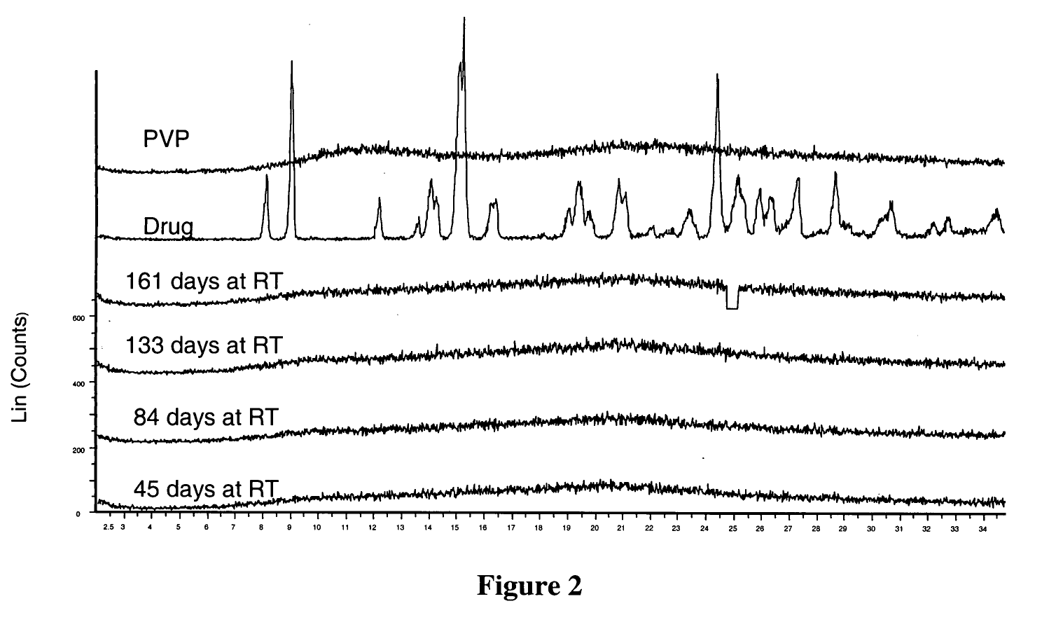

In vitro dissolution rates of samples 1.1, 1.2 and 1.3 were determined using the protocol described in the experimental section. The dissolution medium was a mixture of water and acetonitrile (8:2), and the wavelength used for drug detection 275 nm. Two different lots of unmilled Compound I were also used for comparison. The data shown in FIG. 2, indicates that the electrospun fibers have much higher dissolution rates than the crystalline drug.

The percentage drug dissolved at various time points are collated in the following table, Table 2.

TABLE 2

| | | % Drug Dissolved | | | |
|---|---|---|---|---|---|
| Sample | Drug Content | 10 min | 20 min | 30 min | 40 min |
| Compound I | 99.5% | 17.4 | 24.3 | 29.4 | 33.8 |
| Compound I | | 12.1 | 18.2 | 23.2 | 27.8 |
| Sample 1.1 | 37.3 | 61.1 | 73.5 | 82 | 87.1 |
| Sample 1.2 | 37.1 | 52.4 | 67.7 | 78.5 | 84.1 |
| Sample 1.3 | 33.1 | 36.7 | 61.5 | 73.7 | 82 |

### EXAMPLE 2

### Preparation of amorphous Talnetant (Compound II) by electrospinning

Talnetant HCl, (3-Hydroxy-2-phenyl-N-[(1S)-1-phenylpropyl]-4-quinolinecarboxamide monohydrochloride, also referred to as Compound II, is dissolved in a minimum amount of tetrahydrofuran (THF), and then requisite quantity of PVP and ethanol are added to form a clear yellow solution. This solution is electrospun using the set up. The fibers collected are yellowish in color. Different samples prepared are described in the following table, Table 3.

TABLE 3

| | Sample | Sample | Sample | Sample | Sample | Sample | Sample | Sample | Sample |
|---|---|---|---|---|---|---|---|---|---|
| Ingredients | 2.1 | 2.2 | 2.3 | 2.4 | 2.5 | 2.6 | 2.7 | 2.8 | 2.9 |
| Compound II | 400 mg | 400 | 400 | 2 g | 1 g | 2 g | 400 mg | 600 mg | 600 mg |
| THF | 2 ml | 2 ml | 2 ml | 5 ml | 2.5 ml | 5 ml | 1.4 ml | 2.1 ml | 2.1 ml |
| PVP | 600 mg | 550 mg | 550 | 3 g | none | none | 550 mg | 860 mg | 860 mg |
| Kolloidon | none | none | none | none | 1.5 g | 3 g | none | none | none |
| VA64 | | | | | | | | | |
| Ethanol | 10 ml | 10 ml | 10 ml | 50 ml | 10 ml | 20 ml | 10 ml | 13 ml | 13 ml |
| Surfactant | none | Tween 80/ | TPGS/ | none | none | none | Tween | none | none |
| | | 50 mg | 50 mg | | | | 80/50 mg | | |
| Yield | 900 mg | 850 mg | 860 mg | 3.8 g | 2.3 g | 4.4 g | 720 mg | 1065 mg | 1065 mg |
| Drug content | 36.7% | 36.6% | 39.9% | 40.7% | 40.% | 39.1% | 39.23% | 41.12% | 38.68% |
| by HPLC | | | | | | | | | |

XRPD of the Electrospun Compound II, Sample 2.1

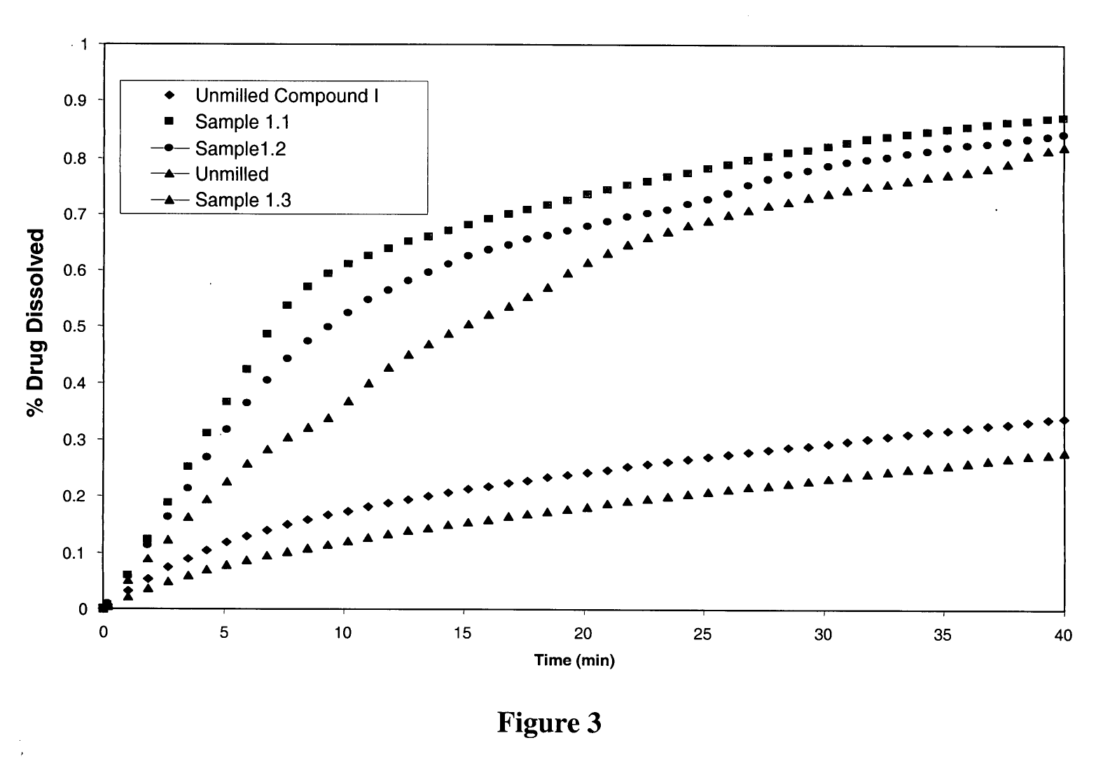

XRPDs of the electrospun sample 2.1 after storage at 25° C. and zero humidity for several days up to 161 days, show the sample to be amorphous. FIG. 3 compares the XRPDs of sample 1.2 stored for 4, 43, and 120 days, along the XRPD of crystalline drug and PVP.

Thermal Analysis of Samples 2.1, 2.2, 2.3, and 2.4

Crystalline Compound II exhibits crystalline melting endotherm at 161° C., whereas the electrospun samples 2.1, 2.2, 2.3 and 2.4 do not have a crystalline melting endotherm, when heated from 0 to 200° C.

MDSC Analysis of Sample 2.7 and 2.8

Analysis confirmed the drug to be in an amorphous state.

In vitro Dissolution Rates

In vitro dissolution rates of samples 2.1, 2.2, 2.3, 2.4, 2.5 and 2.6 were determined using the protocol described in the experimental section. The dissolution medium was 0.1M HCl, and the wavelength used for drug detection 244 nm. An unmilled lot of Compound II was used for comparison. As shown in Table 4 below, the electrospun formulations have much faster rate of dissolution.

TABLE 4

| | | % Drug Dissolved | | | |
|---|---|---|---|---|---|
| Sample | Drug Content | 10 min | 20 min | 30 min | 40 min |
| Compound II | 99.5% | 3.8 | 6.3 | 8.5 | 10.7 |
| Sample 2.1 | 36.7 | 15.7 | 30.1 | 43.8 | 59.1 |
| Sample 2.2 | 36.6 | 24.8 | 42.6 | 58.8 | 69.9 |
| Sample 2.3 | 39.9 | 19.6 | 44.9 | 62.8 | 75.9 |
| Sample 2.4 | 40.7 | 8.5 | 15.1 | 21.1 | 29.8 |
| Sample 2.5 | 40. | 19.8 | 31.1 | 41.1 | 50.1 |
| Sample 2.6 | 39.1 | 26.2 | 40.2 | 52.0 | 60.3 |

### EXAMPLE 3

### Preparation of amorphous formulations of various drugs

Various drugs such as rosiglitazone maleate (Avandia®), eprosartan, carvedilol (Coreg®), hydrochloridethiazide, aspirin, naproxen, nifedipine, indomethacin, and ketoprofen were solubilized in appropriate solvents and mixed with PVP dissolved in ethanol to form clear solutions. These solutions were electrospun using the set up described in the experimental section above, and fibers containing the amorphous drug were collected. The following table, Table 5 describes the various formulations used to prepare the electrospun samples.

TABLE 5

| | Amount of | | | | | | Amorphous | |
|---|---|---|---|---|---|---|---|---|
| Drug | drug | Solvent | PVP | | Ethanol | Yield | DSC | XRPD |
| rosiglitazone | 350 mg | THF/8 ml | 550 | mg | none | poor | yes | yes |
| rosiglitazone | 350 mg | DCM*/ | 550 | mg | 9 ml | poor | yes | yes |
| | | 3 ml | | | | | | |
| carvedilol | 700 mg | NMP**/ | 1.2 | g | 6 ml | 0.3 g | yes | yes |
| | | 4 ml | | | | | | |
| eprosartan | 350 mg | NMP/3 ml | 600 | mg | 6 ml | 0.2 g | yes | yes |
| hydrochlorothiazide | 400 mg | Acetone/ | 600 | mg | 5 ml | 0.7 g | yes | yes |
| | | 3 ml | | | | | | |
| aspirin | 800 mg | Ethanol/ | 1.2 | g | 5 ml | 1.8 g | yes | yes |
| | | 10 ml | | | | | | |
| naproxen | 800 mg | Ethanol/ | 1.2 | g | 5 ml | 1.8 g | yes | yes |
| | | 10 ml | | | | | | |
| nifedipine | 800 mg | Ethanol/ | 1.2 | g | 5 ml | 2 g | yes | yes |
| | | 10 ml | | | | | | |
| indomethacin | 800 mg | Aceto-nitri | 1.2 | g | 10 ml | 1.8 g | yes | yes |
| | | 5 ml | | | | | | |

*DCM—Dichloromethane
**NMP—N-methyl pyrrolidone

### EXAMPLE 4

### Electrospinning of 35.52% (w/w) Carvedilol HBr monohydrate composition

400 mg of crystalline material, Carvedilol HBr monohydrate was dissolved in 4.0 mL of tetrahydrofuran (Mallinckrodt) and 3 mL of MilliQ™ water. The drug solution was added to 600 mg of POLYOX WSR 1105 (Union Carbide) in 10 mL of acetonitrile (EM). The contents were mixed to form a solution. This polymer solution has 1441 μS/cm of conductivity and 676 Cp of viscosity. This solution was spun using similar conditions as described above in Example 4 above to yield 402 mg of nanofibers containing the title compound. The morphology of the drug using MDSC was confirmed as amorphous. Over time, the morphology of the drug will convert to a crystalline form.

### EXAMPLE 5

### Electrospinning of (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate-39.76% (w/w) composition

400 mg of the free base, crystalline form title compound was dissolved in 2.0 mL of methylene chloride (EM) The drug solution was added to 600 mg of Eudragit L100-55 (Rohm) in 2.0 mL of ethanol (AAPER). This solution was spun using similar conditions as described above in Example 2, above to yield 340 mg of nanofibers containing the compound. The morphology of the drug using MDSC was confirmed as amorphous.

### EXAMPLE 6

### Electrospinning of 37.58% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

500 mg of the title compound (crystalline form, free base) was dissolved in 2.5 mL of methylene chloride (EM) The drug solution was added to 700 mg of POLYOX WSR 1105 (Union Carbide) in 15 mL of acetonitrile (EM). 50 mg of Tween 80 (J. T. Baker) was added and polymer solution was clear. This solution was electrospun using similar conditions as described above in Example 2, above, to yield 774 mg of nanofibers containing the title compound. The morphology of the drug using MDSC and X-Ray diffraction was confirmed as crystalline.

Repeat synthesis of the fibers using the conditions set forth in this example yielded a drug load of 39.12% w/w, and 38.06%, respectively and the morphology determination by MDSC, and XRD as crystalline.

### EXAMPLE 7

### Electrospinning of 30.22% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

400 mg of the title compound (76.46%, tosylate salt) as an amorphous form, was dissolved in 3.0 mL of methylene chloride (EM) The drug solution was added to 600 mg of Eudragit L100-55 (Rohm) in 3.0 mL of ethanol (AAPER). 10 mg of Tween 80 (J. T. Baker) was added to the solution. This solution was electrospun using similar conditions as described above in Example 2, above, to yield 224 mg of nanofibers containing the compound. The morphology of the drug in the spun fiber using MDSC and X-Ray diffraction was confirmed as amorphous.

A repeat of this experiment yielded a drug content of 29.66% w/w and confirmed morphology using MDSC and X-Ray diffraction as amorphous.

### EXAMPLE 8

### Electrospinning of 29.66% (w/w) (−)-(S)-N-[α-Ethylbenzyl)-3-hydroxy-2-phenyl quinoline-4-carboxamide HCl composition

600 mg of the title compound was dissolved in 2.1 mL of tetrahydrofuran (Aldrich). The drug solution was added to 1030 mg of POLYOX WSR 1105 (Union Carbide) in 26 mL of acetonitrile (EM) together with 80 mg of Tween 80 (J. T. Baker). The contents were mixed to form a solution, then the polymer solution was sonicated for fifteen minutes. The solution was electrospun using similar conditions as described above in Example 2, above to yield 636 mg of nanofibers containing the title compound. The morphology of the drug using MDSC and X-ray Diffraction was confirmed as crystalline.

### EXAMPLE 9

### Electrospinning of 29.86% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (Tosylate) composition

400 mg of the title compound as the amorphous form, tosylate salt (strength 78.74%) was dissolved in 2.0 mL of methylene chloride (EM). The drug solution was added to 600 mg of POLYOX WSR 1105 (Union Carbide) in 23 mL of acetonitrile (EM) together with 60 mg of Tween 80 (J. T. Baker). The contents were mixed to form a solution. The solution was electrospun using similar conditions as described above in Example 2 above, to yield 339 mg of nanofibers containing the compound. The morphology of the drug using MDSC and X-Ray diffraction was confirmed as amorphous.

### EXAMPLE 10

### Electrospinning of 29.08% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

800 mg of the title compound (crystalline form) was completely dissolved in 5.0 mL of methylene chloride (EM). 1300 mg of polycaprolactone(hereinafter “PCL”) and 400 mg of POLYOX WSR 1105 (Union Carbide) were added into drug solution together with 1 mL of acetonitrile (EM). The contents were mixed to form a solution. The solution was electrospun using similar conditions as described above in Example 2, above. 757 mg of nanofibers containing the compound were collected. The morphology of the drug substance as determined by MDSC was crystalline.

### EXAMPLE 11

### Electrospinning of 48.46% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

800 mg of the title compound (crystalline form) was completely dissolved in 5.0 mL of methylene chloride (EM). 800 mg of PCL was added into drug solution together with additional 3.0 mL of methylene chloride (EM). The contents were mixed to form a solution. The solution was electrospun using similar conditions as described above in Example 2, above. 482 mg of nanofibers containing the compound were collected from the drum. The morphology of the drug substance as determined by MDSC was crystalline.

### EXAMPLE 12

### Electrospinning of 39.14% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (Tosylate) composition

1000 mg of the title compound (amorphous form) was completely dissolved in 3.0 mL of methylene chloride (EM). The drug solution was added into 500 mg of PCL and 500 mg of POLYOX WSR 1105 (Union Carbide) in 13 mL of acetonitrile (EM) The resultant solution was electrospun using conditions similar to Example 2 above, but using a feed pressure of 1 psi. 1.5524 g of fibers were collected and removed from the drum. The morphology of the drug substance as determined by MDSC was amorphous.

### EXAMPLE 13

### Electrospinning of 38.35% (w/w) (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

3.0 g of the free base, crystalline form title compound was dissolved in 15.0 mL of methylene chloride (EM) The drug solution was added to 4.5 g of Eudragit L100-55 (Rohm) in 22.0 mL of ethanol (AAPER). After that 98 mg of Tween 80 (J. T. Baker) was added into the polymer solution. This solution was spun using similar conditions as described above in Example 2, above to yield 5.2 g of nanofibers containing the compound. The morphology of the drug substance as determined by MDSC was amorphous.

### EXAMPLE 14

### Electrospinning of ˜40% (w/w) 3-methyl-N-[(1S)-3-methyl-1-({[(4S,7R)-7-methyl-3-oxo-1-(2-pyridinylsulfonyl)hexahydro-1H-azepin-4-yl]amino}carbonyl)butyl]furo[3,2-b]pyridine-2-carboxamide composition

400 mg of the title compound, as an amorphous material was dissolved in 1.8 mL of tetrahydrofuran (Aldrich). The drug solution was added to 600 mg of POLY OX WSR 1105 (Union Carbide) in 16 mL of acetonitrile (EM). This solution was electrospun using similar conditions as described above in Example 2, to yield 85 mg of nanofibers containing the title compound. The morphology of the drug substance as determined by MDSC was amorphous.

General Experimental for Amorphous Forms and Compositions of Rosiglitazone and Salts Thereof

X-Ray Powder Diffractogram patterns (XRPD's) were recorded on a Phillips PW1730/10 spectrometer using the following acquisition conditions: Tube anode: Cu, Start angle: 4.0°2θ, End angle: 35.0°2θ, Step size: 0.05°2θ, Time per step: 1.0 second.

### EXAMPLE 15

### Preparation of a 1:4 wt:wt rosiglitazone/hydroxypropylmethyl cellulose solid dispersion.

Rosiglitazone (4 g) was added to hydroxypropylmethyl cellulose (16 g) in a mixture of tetrahydrofuran (240 ml) and methanol (60 ml) and the mixture was stirred at ambient temperature until the solid dissolved. The solution was spray-dried using a Niro SDMicro under the following conditions:

| | |
|---|---|
| Process Flow | 20 Kg/h |
| Inlet Temperature | 62° C. |
| Nozzle Flow | 5.0 Kg/h |
| Nozzle φ | 0.5 mm |
| Nozzle Position | 0.5 mm |

12.0 g of product was collected from the cyclone. The product was dried at 40° C. under vacuum for 24 hours to afford the solid dispersion as a fine, white powder.

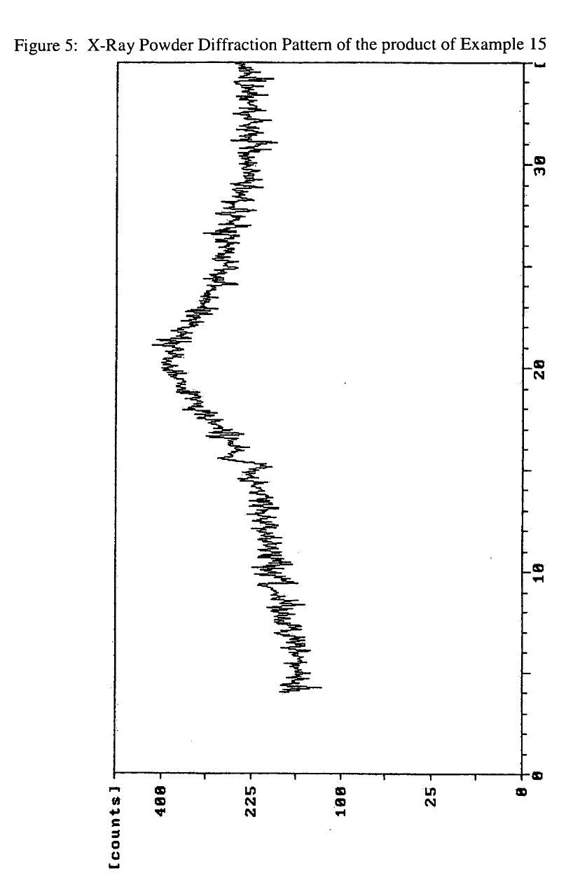

An XRPD confirmed the product was amorphous (see FIG. 5).

### EXAMPLE 16

### Preparation of 1:4 wt:wt rosiglitazone/hydroxypropyl cellulose solid dispersion

Rosiglitazone (4 g) was added to a solution of hydroxypropyl cellulose (16 g) in tetrahydrofuran (300 ml) and the mixture was stirred at ambient temperature until the solid dissolved. The solution was spray-dried using a Niro SDMicro under the following conditions:

| | |
|---|---|
| Process Flow | 20 Kg/h |
| Inlet Temperature | 65° C. |
| Nozzle Flow | 5.0 Kg/h |
| Nozzle φ | 0.5 mm |
| Nozzle Position | 0.5 mm |

10.2 g of product was collected from the cyclone. The product was dried at 40° C. under vacuum for 24 hours to give the solid dispersion as a fine, white powder.

An XRPD confirmed the product was amorphous.

### EXAMPLE 17

### Preparation of 1:2 wt:wt rosiglitazone/ethyl cellulose solid dispersion

Rosiglitazone (5 g) was added to a solution of ethyl cellulose (10 g) in tetrahydrofuran (300 ml). The mixture was stirred at ambient temperature until the solid dissolved. The solution was spray-dried using a Niro SDMicro under the following conditions:

| | |
|---|---|
| Process Flow | 20 Kg/h |
| Inlet Temperature | 65° C. |
| Nozzle Flow | 5.0 Kg/h |
| Nozzle φ | 0.5 mm |
| Nozzle Position | 0.5 mm |

9.6 g of product was collected from the cyclone. The product was dried at 40° C. under vacuum for 24 hours to afford the solid dispersion as a fine powder.

An XRPD confirmed the product was amorphous.

### EXAMPLE 18

### Preparation of 1:2 wt:wt rosiglitazone/polymethyl methacrylate solid dispersion

Rosiglitazone (5 g) was added to a solution of polymethyl methacrylate (10 g) in a mixture of dichloromethane (200 ml) and tetrahydrofuran (100 ml) and the solid dissolved at ambient temperature. The solution was spray-dried using a Niro SDMicro under the following conditions:

| | |
|---|---|
| Process Flow | 20 Kg/h |
| Inlet Temperature | 65° C. |
| Nozzle Flow | 5.0 Kg/h |
| Nozzle φ | 0.5 mm |
| Nozzle Position | 0.5 mm |

9.2 g of product was collected from the cyclone. The product was dried at 40° C. under vacuum for 24 hours to produce the solid dispersion as a fine powder.

An XRPD confirmed the product was amorphous.

### EXAMPLE 19

### Preparation of amorphous rosiglitazone maleate

Rosiglitazone maleate (1 g) was suspended in methanol (10 ml) and the mixture was warmed gently to 40° C. to dissolve the solid. The clear solution was filtered and the filtrate was concentrated on a rotor evaporator at 40° C. to give a light and fluffy solid residue.

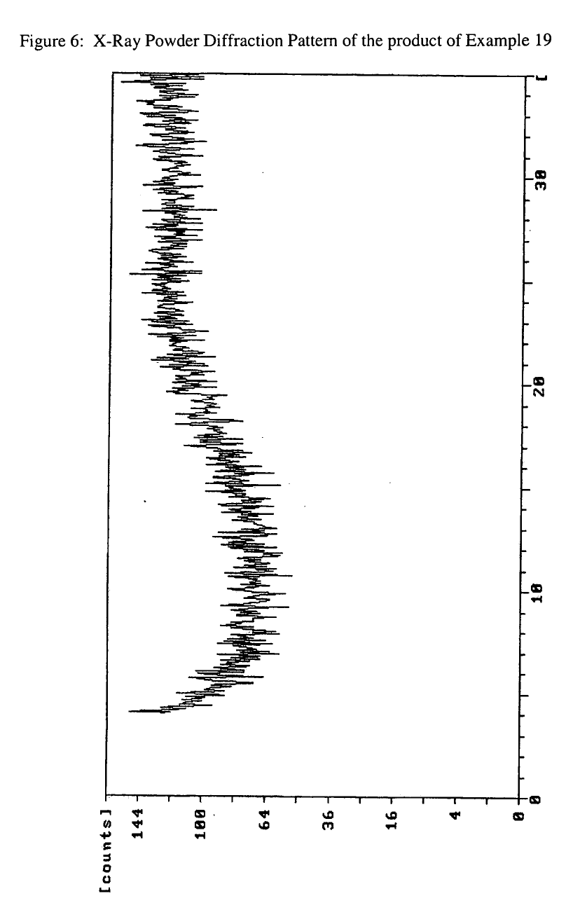

An XRPD confirmed the product was amorphous (see FIG. 6).

### EXAMPLE 20

### Preparation of amorphous rosiglitazone maleate.

Rosiglitazone maleate (25 g) was dissolved in a mixture of methanol (150 ml) and acetone (150 ml) at ambient temperature. The solution was spray-dried using a Niro SDMicro under the following conditions:

| | |
|---|---|
| Process Flow | 20 Kg/h |
| Inlet Temperature | 62° C. |
| Nozzle Flow | 5.0 Kg/h |
| Nozzle orifice (φ) | 0.5 mm |
| Nozzle Position | 0.5 mm |

7 g of product was collected from the cyclone. The solid was dried at 40° C. under vacuum overnight to give amorphous rosiglitazone maleate as a fine powder.

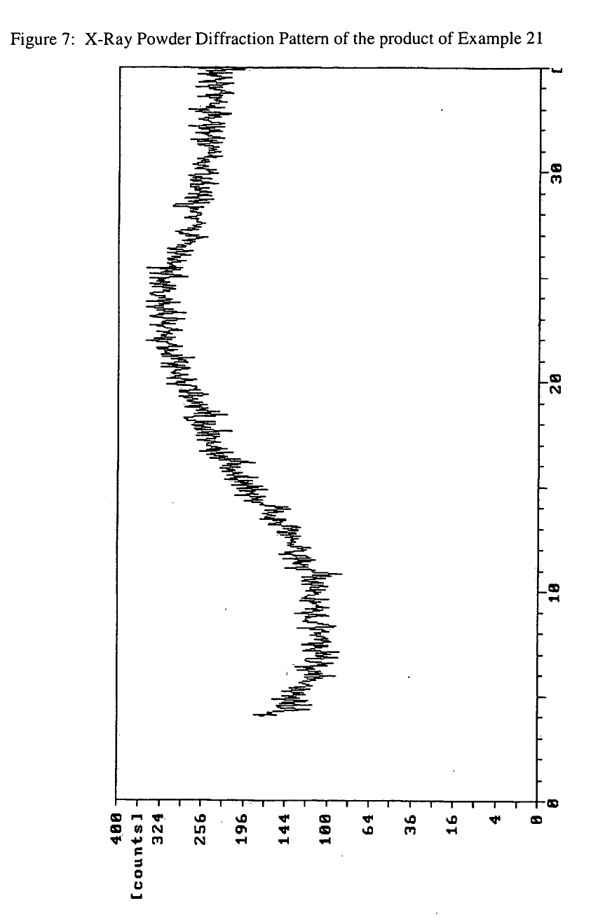

An XRPD confirmed the product was amorphous (see FIG. 7).

### EXAMPLE 21

### Preparation of 1:2 wt:wt rosiglitazone maleate/HPMC solid dispersion

Rosiglitazone maleate (10 g) was added to hydroxypropylmethyl cellulose (20 g) in a mixture of methanol (450 ml) and water (150 ml) and the mixture was stirred at ambient temperature until the solid dissolved. The solution was spray-dried using a Niro SDMicro under the following conditions:

| | |
|---|---|
| Process Flow | 20 Kg/h |
| Inlet Temperature | 62° C. |
| Nozzle Flow | 5.0 Kg/h |
| Nozzle φ | 0.5 mm |
| Nozzle Position | 0.5 mm |

12.9 g product was collected from the cyclone. A further 4.6 g was recovered from the filter. The product was dried at 40° C. under vacuum for 24 hours. The product was a very fine, free-flowing powder.

An XRPD confirmed the product was amorphous (see FIG. 8).

### EXAMPLE 22

### Preparation of a 1:2 wt:wt. rosiglitazone maleate/methyl cellulose solid dispersion

Rosiglitazone maleate (5 g) was added to methyl cellulose (10 g) in a mixture of methanol (200 ml) and water (100 ml). The mixture was stirred at ambient temperature until the solid dissolved. The slightly cloudy solution was spray-dried using a Niro SDMicro under the following conditions:

| | |
|---|---|
| Process Flow | 20 Kg/h |
| Inlet Temperature | 81° C. |
| Nozzle Flow | 5.0 Kg/h |
| Nozzle φ | 0.5 mm |
| Nozzle Position | 0.5 mm |

3.9 g product was collected from the cyclone. The product was dried at 40° C. under vacuum for 24 hours. The product was a very fine, free-flowing powder.

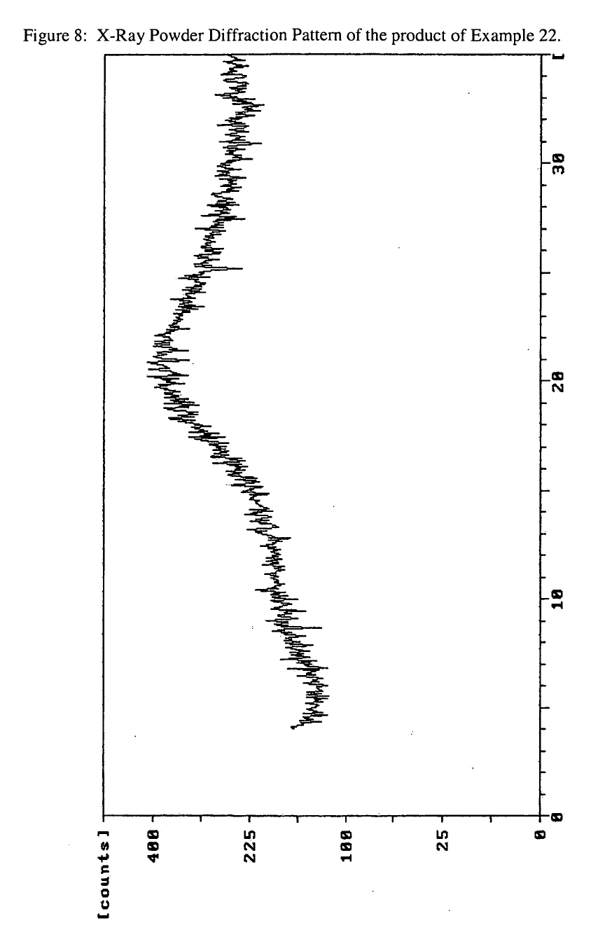

An XRPD confirmed the product was amorphous (see FIG. 9).

### EXAMPLE 23

### Preparation of a 1:2 wt:wt. rosiglitazone maleate/ethyl cellulose solid dispersion.

Rosiglitazone maleate (5 g) was added to ethyl cellulose (10 g) in a mixture of methanol (200 ml) and acetone (100 ml) and the mixture was stirred at ambient temperature until the solid dissolved. The solution was spray-dried using a Niro SDMicro under the following conditions:

| | |
|---|---|
| Process Flow | 20 Kg/h |
| Inlet Temperature | 72° C. |
| Nozzle Flow | 5.0 Kg/h |
| Nozzle φ | 0.5 mm |
| Nozzle Position | 0.25 mm |

9.5 g product was collected from the cyclone. The product was dried at 40° C. under vacuum for 24 hours. The product was a very fine, free-flowing powder.

An XRPD confirmed the product was amorphous.

### EXAMPLE 24

### Preparation of a 1:4 wt:wt. rosiglitazone maleate/HPC solid dispersion

Rosiglitazone maleate (4 g) was added to hydroxypropyl cellulose (16 g) in a mixture of methanol (240 ml) and water (160 ml) and the mixture was stirred at ambient temperature until the solid dissolved. The solution was spray-dried using a Niro SDMicro under the following conditions:

| | |
|---|---|
| Flow | 20 Kg/h |
| Inlet Temperature | 70° C. |
| Nozzle Flow | 5.0 Kg/h |
| Nozzle φ | 0.5 mm |
| Nozzle Position | 0.5 mm |

7.7 g product was collected from the cyclone. The product was dried at 40° C. under vacuum for 24 hours to afford the solid dispersion as a fine powder.

An XRPD confirmed the product was amorphous.

### EXAMPLE 25

### Preparation of a 1:2 wt:wt. rosiglitazone maleate/polymethyl methacrylate solid dispersion

Rosiglitazone maleate (5 g) was added to polymethylmethacrylate (10 g) in a mixture of dichloromethane (120 ml) and methanol (80 ml) and the solid dissolved at ambient temperature. The solution was spray-dried using a Niro SDMicro under the following conditions:

| | |
|---|---|
| Process Flow | 20 Kg/h |
| Inlet Temperature | 65° C. |
| Nozzle Flow | 5.0 Kg/h |
| Nozzle φ | 0.5 mm |
| Nozzle Position | 0.5 mm |

10.5 g product was collected from the cyclone. The product was dried at 40° C. under vacuum for 24 hours to give the solid dispersion as a fine powder.

An XRPD confirmed the product was amorphous.

### EXAMPLE 26

### Preparation of amorphous rosiglitazone hydrochloride

Rosiglitazone hydrochloride dihydrate (1.7 g) was dissolved in water (200 mL) at 21° C. with stirring. The solution was filtered, frozen in a dry ice/acetone bath and the water removed by freeze drying to afford amorphous rosiglitazone hydrochloride as a fluffy white solid.

An XRPD confirmed the product was amorphous.

### EXAMPLE 27

### Preparation of amorphous rosiglitazone hydrochloride

Rosiglitazone hydrochloride (1.0 g) and methanol (5 ml) was heated at reflux for 1 hour to give a clear yellow solution. The solvent was removed under reduced pressure (water bath temp=37° C.) to give a glassy solid, which was dried under vacuum for 2 hour 50 mins at 21° C. to give amorphous rosiglitazone hydrochloride as a powdery solid (0.8 g)

An XRPD confirmed the product was amorphous.

### EXAMPLE 28

### Preparation of amorphous rosiglitazone hydrochloride

Rosiglitazone hydrochloride (25.1 g) and methanol (125 ml) was heated at reflux for 1 hour. The solvent was removed under reduced pressure to give a glassy solid, which was dried under vacuum at 21° C. for 3 hours to give amorphous rosiglitazone hydrochloride as a powdery solid (22.2 g).

An XRPD confirmed the product was amorphous.

### EXAMPLE 29

### Preparation of amorphous rosiglitazone hydrochloride

A solution of rosiglitazone hydrochloride dihydrate (10 g) in methanol (100 ml) was spray-dried using a Niro SDMicro under the following conditions:

| | |
|---|---|
| Flow | 30 Kg/h |
| Inlet Temperature | 60° C. |
| Nozzle Flow | 5.0 Kg/h |
| Nozzle φ | 0.5 mm |
| Nozzle Position | 0.25 mm |

3.4 g product was collected from the cyclone. The sample was dried at 40° C. under vacuum for 24 hours to afford amorphous rosiglitazone hydrochloride as a fine powder.

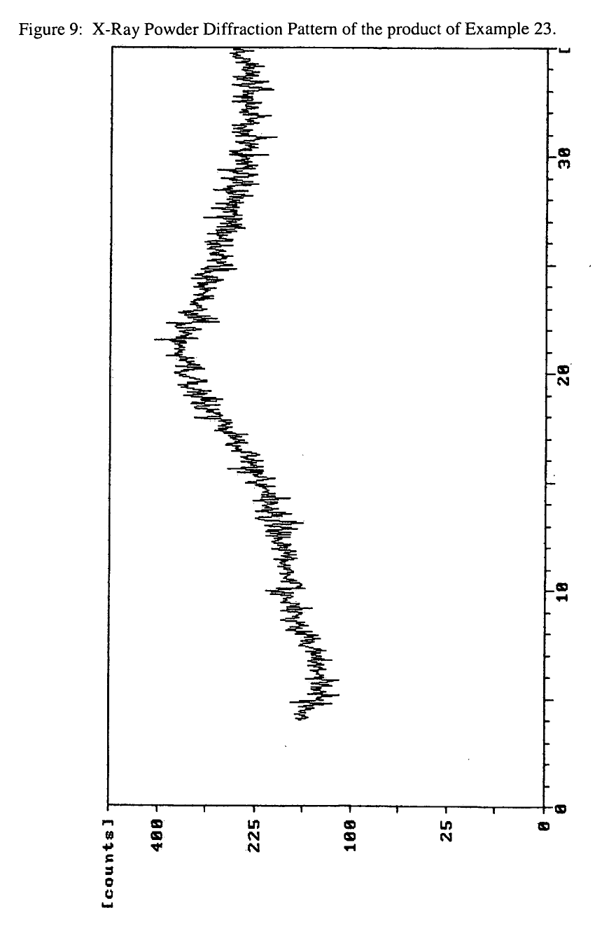

An XRPD confirmed the product was amorphous (see FIG. 10).

### EXAMPLE 30

### Preparation of a 1:2 wt:wt rosiglitazone hydrochloride/PEG solid dispersion

Polyethylene glycol (2 g) was added to a solution of rosiglitazone hydrochloride dihydrate (1 g) in methanol (20 ml) at ambient temperature. The resulting suspension was concentrated under reduced pressure at −50° C. and a white solid residue was obtained which was dried at 50° C. under vacuum for 3-4 hours to afford the solid dispersion as a white solid.

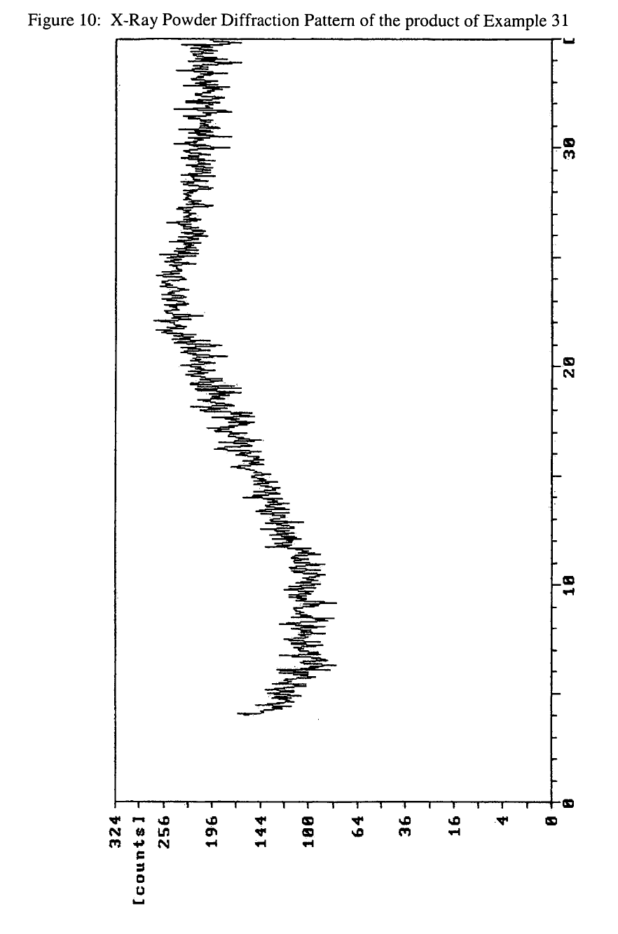

An XRPD confirmed that the rosiglitazone hydrochloride was amorphous. The observed peaks are consistent with polyethylene glycol (see FIG. 11).

### EXAMPLE 31

### Preparation of a 1:2 wt:wt. rosiglitazone hydrochloride/HPMC solid dispersion

Rosiglitazone hydrochloride dihydrate (5 g) was added to a solution of hydroxypropyl methyl cellulose (10 g) in a mixture of methanol (250 ml) and water (50 ml) and stirred at ambient temperature until the solid dissolved. The solution was spray-dried using a Niro SDMicro under the following conditions:

| | |
|---|---|
| Process Flow | 20 Kg/h |
| Inlet Temperature | 65° C. |
| Nozzle Flow | 5.0 Kg/h |
| Nozzle φ | 0.5 mm |
| Nozzle Position | 0.5 mm |

6.5 g product was collected from the cyclone. The sample was dried at 40° C. under vacuum for 24 hours to afford the solid dispersion as a fine powder.

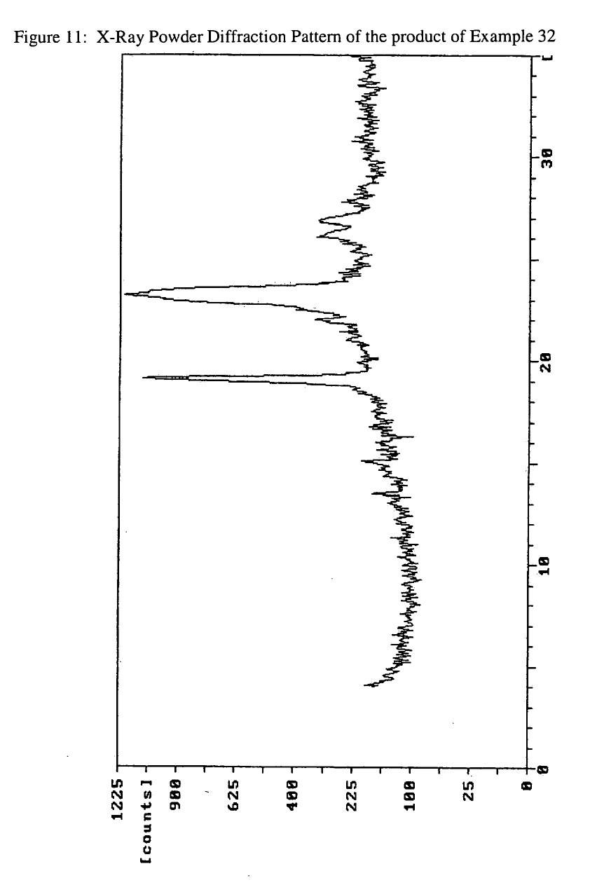

An XRPD confirmed the product was amorphous (see FIG. 12).

### EXAMPLE 32

### Preparation of amorphous rosiglitazone potassium salt

Rosiglitazone potassium salt (5.0 g) was dissolved in water (200 mL). The solution was rapidly frozen in a dry ice/acetone bath before the water was removed by freeze drying to give amorphous rosiglitazone potassium salt as a fluffy white solid.

An XRPD confirmed the product was amorphous.

### EXAMPLE 33

### Preparation of 1:2 wt:wt. rosiglitazone potassium salt/HPMC solid dispersion

Rosiglitazone potassium salt (10 g) was added to a solution of hydroxypropyl methyl cellulose (20 g) in a mixture of acetone (300 ml) and water (100 ml) and stirred ambient temperature until the solid dissolved at. The solution was spray-dried using a Niro SDMicro under the following conditions:

| | |
|---|---|
| Process Flow | 20 Kg/h |
| Inlet Temperature | 55° C. |
| Nozzle Flow | 5.0 Kg/h |
| Nozzle φ | 0.5 mm |
| Nozzle Position | 0.25 mm |

9.1 g product was collected from the cyclone. The sample was dried further at 40° C. under vacuum for 24 hours to give the solid dispersion as a fine powder.

An XRPD confirmed the product was amorphous.

### EXAMPLE 34

### Preparation of a 1:1 wt:wt rosiglitazone potassium salt/HPMC solid dispersion

Rosiglitazone potassium salt (1 g) was added to a solution of hydroxypropyl methyl cellulose (2 g) in a mixture of acetone (10 ml) and water (5 ml) and stirred so that the solid dissolved at ambient temperature. The solution was concentrated under reduced pressure and the residue was dried at 40° C. under vacuum for 24 hours to give the product as a solid.

An XRPD confirmed the product was amorphous.

### EXAMPLE 35

### Preparation of a 1:1 wt:wt rosiglitazone potassium salt/ethyl cellulose solid dispersion

Rosiglitazone potassium salt (1 g) was added to a suspension of ethyl cellulose (2 g) in a mixture of acetone (20 ml) and methanol (40 ml) and stirred at ambient temperature until the solid dissolved. The solution was concentrated under reduced pressure at −40° C. and a solid residue was obtained which was further dried at 40-50° C. under vacuum for 24 hours to afford the product as a solid.

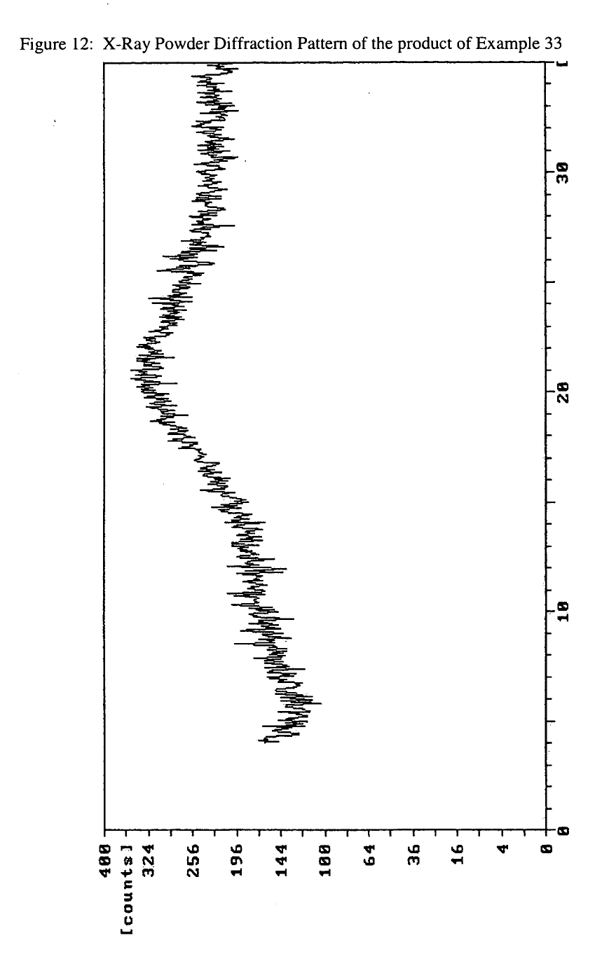

An XRPD confirmed the product was amorphous (see FIG. 13).

### EXAMPLE 36

### Preparation of amorphous rosiglitazone mesylate

Rosiglitazone mesylate (2 g) was dissolved in a mixture of propan-2-ol (20 ml) and water (10 ml) at ambient temperature. The solution was concentrated on a rotary evaporator at 40° C. to give an off-white solid. This was dried further at 40° C. under vacuum for 24 hours to give amorphous rosiglitazone mesylate as an off white solid.

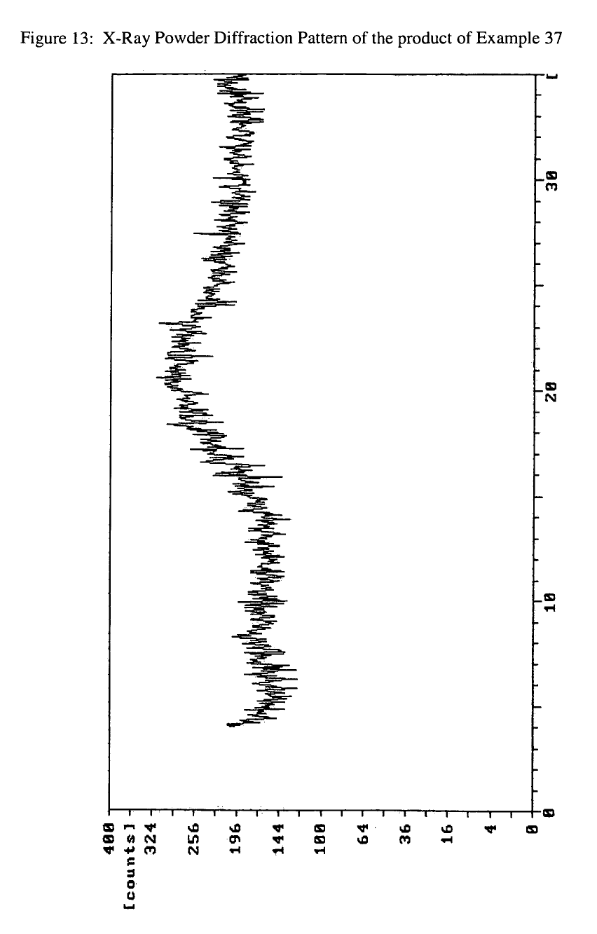

An XRPD confirmed the product was amorphous (see FIG. 14).

### EXAMPLE 37

### Preparation of amorphous rosiglitazone mesylate

A 5.0 wt. % solution of rosiglitazone mesylate in 90/10 wt. % acetone/water was spray dried using a Niro SDMicro under the following conditions:

Inlet temperature: 153° C.

Outlet temperature: 89° C.

Nitrogen flow rate: 15 kg/h.

Feed flow rate: 615 g/h.

Nitrogen/feed ratio: 4.5.

3.8 g of rosiglitazone mesylate was recovered from under the cyclone as a powder.

### EXAMPLE 38

### Preparation of amorphous rosiglitazone mesylate

A 3.1 wt. % solution of rosiglitazone mesylate in 89/11 wt. % propan-2-ol/water was spray dried using a Niro SDMicro under the following conditions:

Inlet temperature: 141° C.

Outlet temperature: 93° C.

Nitrogen flow rate: 23 kg/h.

Feed flow rate: 360 g/h.

Nitrogen/feed ratio: 8.6.

4.0 g of amorphous rosiglitazone mesylate was recovered as a powder.

### EXAMPLE 39

### Preparation of a 1:2 wt:wt rosiglitazone mesylate/HPMC solid dispersion

Rosiglitazone mesylate (1 g) was added to a suspension of hydroxypropyl methyl cellulose (2 g) in a mixture of propan-2-ol (20 ml) and water (20 ml) and stirred at ambient temperature until solid dissolved. The solution was concentrated under reduced pressure at −40° C. and the residue obtained was dried at 40-50° C. under vacuum for 24 hours to give the product as a solid.

An XRPD confirmed the product was amorphous.

### EXAMPLE 40

### Preparation of a 1:2 wt:wt rosiglitazone mesylate/ethyl cellulose solid dispersion

Rosiglitazone mesylate (1 g) was added to a solution of ethyl cellulose (2 g) in a mixture of methanol (40 ml) and acetone (20 ml) and stirred at ambient temperature until the solid dissolved. The solution was concentrated under reduced pressure at ˜40° C. and a solid residue was obtained which was further dried at 40-50° C. under vacuum for several hours to afford the product as a solid.

An XRPD confirmed the product was amorphous.

### EXAMPLE 41

### Preparation of a 1:2 wt:wt rosiglitazone L(+)-tartrate/HPMC solid dispersion

A suspension of hydroxypropyl methyl cellulose (2 g) in a mixture of propan-2-ol (40 ml) and water (20 ml) was heated to reflux and stirred at that temperature until a clear solution was formed. Rosiglitazone L(+)-tartrate (1 g) was added and the solid dissolved rapidly. The solution was allowed to cool to ambient temperature then concentrated under reduced pressure at 50-60° C. A solid residue was obtained which was further dried at 40° C. under vacuum for 24 hours to give the product as a solid.

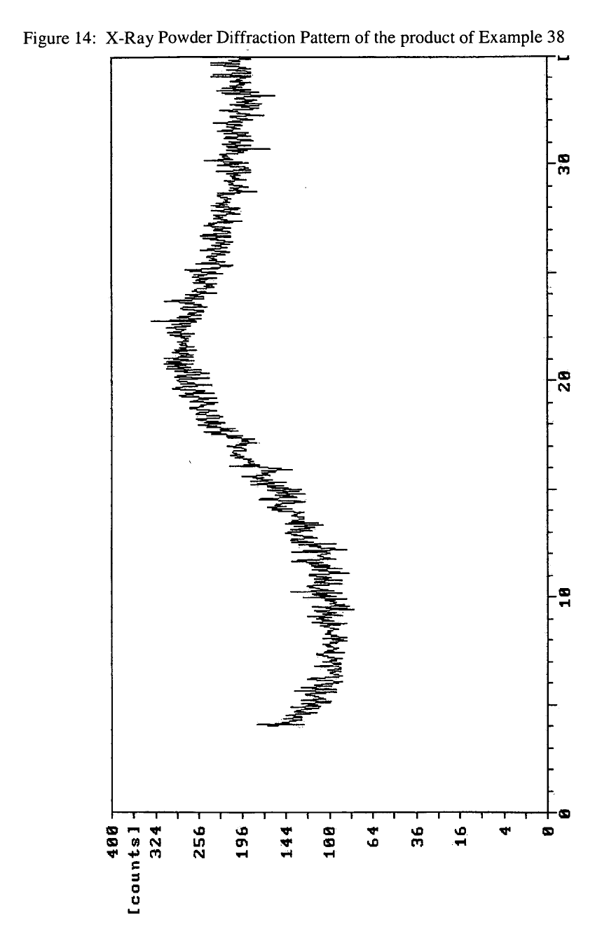

An XRPD confirmed the product was amorphous (see FIG. 15).

### EXAMPLE 42

### Preparation of amorphous rosiglitazone hydrobromide

Rosiglitazone hydrobromide (25.0 g) and methanol (350 ml) was heated at reflux for 2 hours. The hot clear solution was filtered and the filtrate concentrated under reduced pressure to give a glassy material. The product was dried under vacuum, over phosphorus pentoxide for 17 hours at 21° C. to give amorphous rosiglitazone hydrobromide (23.9 g).

An XRPD confirmed the product was amorphous.

### EXAMPLE 43

### Preparation of amorphous rosiglitazone hydrobromide

Rosiglitazone hydrobromide (10 g) was stirred in water (1200 mL) at 21° C. After stirring for 1 hour the solution was filtered, frozen and the solvent was removed by freeze drying to afford amorphous rosiglitazone hydrobromide.

An XRPD confirmed the product was amorphous.

### EXAMPLE 44

### Preparation of a 1:2 wt:wt rosiglitazone/HPMC solid dispersion

Rosiglitazone (1 g) was added to a solution of hydroxypropyl methyl cellulose (2 g) in a mixture of tetrahydrofuran (36 ml) and water (4 ml) and stirred so that the solid dissolved at ambient temperature. The solution was concentrated under reduced pressure (water bath temperature=50° C.) and the oily residue was dried at 40° C. under vacuum for 72 hours to give the product as a solid.

An XRPD confirmed the product was amorphous.

### EXAMPLE 45

### Preparation of a 1:2 wt:wt rosiglitazone/methyl cellulose solid dispersion

Rosiglitazone (1 g) was added to a suspension of methyl cellulose (2 g) in a mixture of tetrahydrofuran (50 ml) and water (10 ml) and stirred well for several minutes so that the oily solid was dispersed. The slightly cloudy mixture was concentrated under reduced pressure (water bath temperature=50° C.) and the white solid residue was dried at 50° C. under vacuum for 48 hours.

An XRPD confirmed the product was amorphous.

### EXAMPLE 46

### Preparation of amorphous rosiglitazone maleate, hydroxypropyl methyl cellulose composition

Amorphous rosiglitazone maleate (1 g) was mixed with hydroxypropyl methyl cellulose (2 g) using a mortar and pestel. The resulting solid was stored in a closed glass vial for 72 hours.

An XRPD was run which indicated the sample was amorphous.

Amorphous rosiglitazone maleate (1 g) was mixed with methyl cellulose (2 g) using a mortar and pestel. The resulting solid was stored in a closed glass vial for 72 hours.

An XRPD was run which indicated the sample was amorphous.

### EXAMPLE 47

### Preparation of amorphous rosiglitazone maleate, PEG, lactose composition.

Amorphous rosiglitazone maleate (1 g) was mixed with polyethylene glycol 4600 (4 g) using a mortar and pestel. The resulting solid was stored in a closed glass vial for one week.

An XRPD was run which showed no additional peaks except those due to Polyethylene Glycol

Amorphous rosiglitazone maleate (1 g) was mixed with methyl cellulose (2 g) using a mortar and pestel. The resulting solid was stored in a closed glass vial for 72 hours.

An XRPD was run which showed no additional peaks except those due to lactose.

### EXAMPLE 48

### Preparation of amorphous rosiglitazone maleate, cellulose composition.

Microcrystalline cellulose (1 g) was added to a solution of rosiglitazone maleate (1 g) in a mixture of methanol (15 ml) and ethyl acetate (15 ml) at ambient temperature. The resulting suspension was concentrated under vacuum to give a white solid residue. This was dried further at 40-50° C. under vacuum then scraped from the side of the flask.

An XRPD was run which showed no additional peaks except those due to microcrystalline cellulose.

### EXAMPLE 49

The following dispersions were made following the same general methods as described in the previous examples noted above:

a) 1:1 rosiglitazone/HPMC

b) 1:1 rosiglitazone/HPMC (spray dried)

c) 1:2 rosiglitazone/HPMC (spray dried)

d) 1:2 rosiglitazone/HPC (spray dried)

e) 1:1 rosiglitazone maleate/BPMC (spray dried)

f) 1:2 rosiglitazone maleate/HPC (spray dried)

j) 1:2 rosiglitazone maleate/PMMA

### EXAMPLE 50

### Stability testing of compositions of rosiglitazone with pharmaceutically acceptable carriers

General Procedure for Stability Testing:

Approximately 0.5 g of amorphous material was placed into a desiccator at 21° C. and 75% relative humidity. After a period of time the sample was removed and an XRPD was run. The length of time the samples were subjected to the high humidity conditions and the results of the XRPD are described in Table 4.

TABLE 4

| | Example | | |
|---|---|---|---|
| Amorphous Material | No. | Time | XRPD Result |
| 1:1 rosiglitazone/HPMC | 35b | 1 day | Amorphous |
| (spray dried) | | | |
| 1:2 rosiglitazone/HPMC | 32 | 4 weeks | Amorphous |
| 1:4 rosiglitazone/HPMC | 1 | 4 weeks | Amorphous |
| (spray dried) | | | |
| 1:2 rosiglitazone/ethyl cellulose | 3 | 4 weeks | Amorphous |
| (spray dried) | | | |
| 1:2 rosiglitazone/methyl cellulose | 34 | 4 weeks | Amorphous |
| 1:2 rosiglitazone/PMMA | 4 | 4 weeks | Amorphous |
| (spray dried) | | | |
| 1:1 rosiglitazone maleate/HPMC | 35j | 1 day | Amorphous |
| (spray dried) | | | |
| 1:2 rosiglitazone maleate/HPMC | 8 | 4 weeks | Amorphous |
| (spray dried) | | | |
| 1:2 rosiglitazone maleate/ethyl | 10 | 4 weeks | Amorphous |
| cellulose (spray dried) | | | |
| 1:2 rosiglitazone maleate/methyl | 9 | 4 weeks | Amorphous |
| cellulose (spray dried) | | | |
| 1:2 rosiglitazone maleate/PMMA | 12 | 4 weeks | Amorphous |
| (spray dried) | | | |

Solubility

Procedure: Small portions of a weighed sample were added to a known volume (30-100 ml) of buffer solution at ambient temperature until solid remained present after a period of stirring (5-10 minutes) as determined by visual inspection. The sample was re-weighed to determine the amount of solid added, from which the approximate solubility in mg/ml was determined.

In pH 4.0 Buffer

1:2 Rosiglitazone maleate/hpmc solid dispersion: ˜5 mg/mL

Amorphous rosiglitazone maleate: ˜0.77 mg/ml

Crystalline rosiglitazone maleate: ˜0.36 mg/mL

In pH 7.0 Buffer

1:2 Rosiglitazone maleate/hpmc solid dispersion: 0.88 mg/mL

Amorphous rosiglitazone maleate: ˜0.5 mg/ml

Crystalline rosiglitazone maleate: ˜0.01 mg/mL

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. Amorphous rosiglitazone.

2. A pharmaceutically acceptable salt of amorphous rosiglitazone which is selected from amorphous rosiglitazone maleate, amorphous rosiglitazone hydrochloride, amorphous rosiglitazone potassium salt, amorphous rosiglitazone mesylate, amorphous rosiglitazone tartrate, or amorphous rosiglitazone hydrobromide.

3. A pharmaceutical composition comprising amorphous rosiglitazone or a pharmaceutically acceptable salt thereof, and a pharmaceutically acceptable carrier.

4. A composition according to claim 3 which is a solid dispersion.

5. A composition according to claim 3, wherein the pharmaceutically acceptable carrier is a polymeric carrier selected from hydroxypropyl methyl cellulose (HPMC), hydroxypropyl cellulose (HPC), methyl cellulose, ethyl cellulose, carboxymethyl cellulose, sodium carboxymethyl cellulose, cellulose acetate phthalate, cellulose acetate butyrate, or hydroxyethyl cellulose; polymethyl methacrylate (PMMA); polymethacrylate; polyvinyl alcohol; polypropylene; polvinylpyrollidone (PVP); polyethylene glycol (PEG); dextrans; dextrins; chitosan; co(lactic/glycolid) copolymers; poly(orthoester); poly(anhydrate); polyvinyl chloride; polyvinyl acetate; ethylene vinyl acetate; lectins; carbopols; silicon elastomers; polyacrylic polymers; maltodextrins; lactose; fructose; inositol; trehalose; maltose; raffinose; or a mixture thereof.

6. The composition according to claim 4 wherein the polymeric carrier is hydroxypropylmethyl cellulose, methyl cellulose, or ethyl cellulose, or a suitable mixture thereof.

7. The composition according to claim 3 wherein the ratio or amorphous rosiglitazone or pharmaceutically acceptable salt thereof to polymeric carrier is:
1:2 wt:wt rosiglitazone/hydroxypropylmethyl cellulose;
1:4 wt:wt rosiglitazone/hydroxypropylmethyl cellulose;
1:2 wt:wt rosiglitazone/methyl cellulose;
1:2 wt:wt rosiglitazone/ethyl cellulose;
1:2 wt:wt rosiglitazone/PMMA;
1:2 wt:wt rosiglitazone maleate/ hydroxypropylmethyl cellulose;
1:2 wt:wt. rosiglitazone maleate/methyl cellulose;
1:2 wt:wt. rosiglitazone maleate/ethyl cellulose;
1:2 wt:wt. rosiglitazone hydrochloride/hydroxypropylmethyl cellulose;
1:2 wt:wt. rosiglitazone potassium salt/hydroxypropylmethyl cellulose;
1:1 wt:wt rosiglitazone potassium salt/ethyl cellulose;
1:2 wt:wt rosiglitazone mesylate/ hydroxypropylmethyl cellulose;
1:2 wt:wt rosiglitazone mesylate/ethyl cellulose; and
1:2 wt:wt rosiglitazone L(+)-tartrate/hydroxypropylmethyl cellulose.

8. A composition according to claim 5 which is a solid dispersion.

9. A pharmaceutical composition comprising anrelectrospun fiber of a pharmaceutically acceptable amorphous polymeric carrier homogeneously integrated with a stable amorphous form of a pharmaceutically acceptable active agent which is rosiglitazone or a pharmaceutically acceptable salt thereof.

10. The composition according to claim 9 wherein the active agent is nanoparticle in size.

11. The composition according to claim 9 wherein the polymeric carrier is water soluble.

12. The composition according to claim 9 wherein the polymeric carrier is water insoluble.

13. The composition according to claim 9 wherein the composition further comprises a surfactant which is a block copolymer of ethylene oxide and propylene oxide, lecithin, sodium dioctyl sulfosuccinate, sodium lauryl sulfate, Tween 20, 60 & 80, Span™, Arlacel™, Triton X-200, polyethylene glycol, glyceryl monostearate, d-alpha-tocopheryl polyethylene glycol 1000 succinate, sucrose fatty acid ester, such as sucrose stearate, sucrose oleate, sucrose palmitate, sucrose laurate, sucrose acetate butyrate, or mixtures thereof.

14. The composition according to claim 13 wherein the surfactant is present in an amount of 0 to about 15% w/w.

15. The composition according to claim 9 wherein the composition further comprises an absorption enhancer.

16. The composition according to claim 9 wherein the polymeric carrier is polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives, poly(alpha-hydroxy acids), poly(alpha-aminoacids) and its copolymers, poly(orthoesters), polyphosphazenes, or poly(phosphoesters).

17. The composition according to claim 16 wherein the polymeric carrier is polyvinyl pyrrolidone or polyvinylpyrrolidone-co-polyvinylacetate.

18. The composition according to claim 16 wherein the polymeric carrier is Eudragit L100-55, Eudragit L30 D55, Eudragit L100, Eudragit S 100, Eudragit E 100, Eudragit EPO, Eudragit RL 30D, Eudragit RL PO, Eudragit RL 100, Eudragit RS 30D, Eudragit RS PO, Eudragit RS 100, Eudragit NE 30, or Eudragit NE 40, or a mixture thereof.

19. The composition according to claim 9 in which active agent is present in an amount of about 1 to about 50% w/w.

20. The composition according to claim 9 in which the active agent demonstrates improved bioavailability and/or improved stability, or has a modified or delayed absorption profile as compared to an immediate release dosage form.

21. The composition according to claim 9 in which the electrospun fiber is encapsulated or compressed into a tablet or capsule.

22. The composition according to claim 9 in which the electrospun fiber is further ground in size.

23. The composition according to claim 9 which is results in a rapid dissolution of the fiber.

24. The composition according to claim 9 which results in controlled release, sustained release, or pulsatile release of the active agent.

25. The composition according to claim 9 which results in immediate release of the active agent.

26. A method for the treatment or prophylaxis of non-insulin dependent diabetes mellitus which comprises administering an effective amount of a composition according to claim 9 to a human in need of said treatment or prophylaxis.

27. A method for the treatment or prophylaxis of non-insulin dependent diabetes mellitus which comprises administering an effective amount of a composition according to claim 3 to a human in need of said treatment or prophylaxis.

28. A process for making amorphous rosiglitazone, or a pharmaceutically acceptable salt thereof, which comprises:
a. dissolving rosiglitazone or a pharmaceutically acceptable salt thereof, in one or more organic solvents, a mixture of organic solvent(s) and water or water; and
b. removing said solvent(s) by evaporation.

29. The process according to claim 28 wherein the organic solvent(s) is selected alcohols, ketones, esters, ethers, nitrites, hydrocarbons, organic acids, and chlorinated solvents or mixtures thereof.

30. The process according to claim 28, wherein the organic solvent(s) are selected from methanol, ethanol, propan-2-ol, acetone, ethyl acetate, tetrahydrofuran, acetonitrile, toluene, acetic acid and dichloromethane or mixtures thereof.

31. The process according to claim 30, wherein the organic solvent is selected from methanol, acetone and tetrahydrofuran or mixtures thereof.

32. The process according to claim 28, wherein step b) is achieved by spray drying.

33. A process for making a composition according to claim 3 which comprises:
a) mixing a solution of rosiglitazone or a pharmaceutically acceptable salt thereof, and a suspension or solution of the pharmaceutically acceptable carrier(s) in one or more organic solvents, a mixture of organic solvent(s) and water or water; and
b) removing said solvent(s) by evaporation.

34. The process according to claim 34, wherein the ratio by weight of the rosiglitazone or a salt thereof to pharmaceutical carrier(s) to is in the range of about 1:33 to about 5:1.

35. The process according to claim 33, wherein the organic solvent(s) is selected alcohols, ketones, esters, ethers, nitrites, hydrocarbons, organic acids and chlorinated solvents or mixtures thereof.

36. The process according to claim 33, wherein the organic solvent(s) are selected from methanol, ethanol, propan-2-ol, acetone, ethyl acetate, tetrahydrofuran, acetonitrile, toluene, acetic acid and dichloromethane or mixtures thereof.

37. The process according to claim 36, wherein the organic solvent is selected from methanol, acetone, dichloromethane and tetrahydrofuran or mixtures thereof.

38. The process according to claim 33, wherein step b) is achieved by spray drying.

39. The process according to claim 33 wherein the pharmaceutically acceptable carrier is a polymeric carrier selected from hydroxypropyl methyl cellulose (HPMC), hydroxypropyl cellulose (HPC), methyl cellulose, ethyl cellulose, carboxymethyl cellulose, sodium carboxymethyl cellulose, cellulose acetate phthalate, cellulose acetate butyrate, or hydroxyethyl cellulose; polymethyl methacrylate (PMMA); polymethacrylate; polyvinyl alcohol; polypropylene; polvinylpyrollidone (PVP); polyethylene glycol (PEG); dextrans; dextrins; chitosan; co(lactic/glycolid) copolymers; poly(orthoester); poly(anhydrate); polyvinyl chloride; polyvinyl acetate; ethylene vinyl acetate; lectins; carbopols; silicon elastomers; polyacrylic polymers; maltodextrins; lactose; fructose; inositol; trehalose; maltose; raffinose; or a mixture thereof.

40. The process according to claim 33 wherein the composition of step a) is precipitated to cause a solid dispersion.

41. The process according to claim 40, wherein the precipitation of the solid dispersion is achieved by addition of an anti-solvent or by changing the pH of the solution.
